(19) United States
(12) Patent Application Publication
(10) Pub. No.: US 2026/0265814 A1
(43) Pub. Date: Sep. 10, 2026

(54) SCALABLE, SUBMICRON-RESOLUTION REPLICATION OF DNA ARRAYS

(71) Applicants: TOPOGENE INC., Seattle, WA (US); UNIVERSITY OF WASHINGTON, Seattle, WA (US)

(72) Inventors: Li SUN, Seattle, WA (US); Liangcai GU, Seattle, WA (US)

(21) Appl. No.: 18/881,374

(22) PCT Filed: Jul. 11, 2023
(86) PCT No.: PCT/US2023/069931

§ 371 Date: Jan. 6, 2025

Related U.S. Application Data
(60) Provisional application No. 63/388,326, filed on Jul. 12, 2022.

(51) Int. Cl. C12Q 1/6844; C12Q 1/48; C12Q 1/6806; C12Q 1/6837; C12Q 1/6841; C12Q 1/6869; C12Q 1/6876; C40B 70/00

(52) U.S. Cl. CPC C12Q 1/6844; C12Q 1/48; C12Q 1/6806; C12Q 1/6837; C12Q 1/6841; C12Q 1/6869; C12Q 1/6876; C12Y 207/07; C40B 70/00; C12Q 2600/156

(57) ABSTRACT

Scalable stamping methods to replicate a template microarray are provided.

### RELATED APPLICATIONS

This application is a 35 U.S.C. § 371 filing of International Patent Application No. PCT/US2023/069931, filed Jul. 11, 2023, which claims the benefit of U.S. Provisional Application No. 63/388,326, filed Jul. 12, 2022, the contents of which are incorporated herein reference in its entirety for all purposes.

### SEQUENCE LISTING

The instant application contains a Sequence Listing which has been submitted electronically in XML format and is hereby incorporated by reference in its entirety. Said XML file, created on Jan. 3, 2025, is named 760466_TOPO-OOTUS_ST26.xml and is 28,389 bytes in size.

### FIELD OF THE INVENTION

The disclosure relates to a vastly scalable stamping method to fabricate DNA arrays.

### BACKGROUND

Nucleic acid microarrays (or DNA arrays) are a class of technologies in which sequence-defined DNAs are deposited (“spotted”) or in situ synthesized in a two-dimensional array on a substrate where the DNAs are covalently or non-covalently attached to the substrate. One array can have hundreds to billions of separated spots (or features) as probes on a glass slide of 75×25 mm² to detect many different target analytes in a highly complex mixture. Thus, they offer a highly parallel, miniaturized assay platform to maximize the throughput and cost-effectiveness. DNA arrays have been widely utilized for the profiling of gene expression and transcription factor binding, genotyping, and DNA synthesis, etc. Most recently, some arrays have been applied to spatial transcriptome and proteomics applications.

Based on their fabrication, DNA arrays can be grouped into three major types: spotted arrays, in-situ synthesized arrays, and self-assembled (or random) arrays. Microarray spotting is the most common method to deposit DNAs, proteins, and other molecules on array substrates. In 1995, Schena et al. developed a method to print complementary DNAs (cDNAs) to monitor the expression of multiple genes on a glass slide in parallel (1). In 1996, DeRisi et al. used a robotic spotter to print >1,000 DNA probes on each poly-lysine coated microscope slide (2). After that, significant advances were made on the spotting devices for contact and non-contact printing to improve the throughput, quality, and reproducibility (3). However, the spotting methods rely on pre-synthesizing and sequentially transferring many different DNAs to substrate surfaces, imposing intrinsic limitations on the cost-effectiveness and throughput of the array fabrication. For example, because non-covalently deposited DNAs on a spot can diffuse to other array locations, it is necessary to use stringent washes to remove unbound DNAs and separate adjacent features with significant gaps to prevent feature merging. The initial feature center-to-center distance was 450 μm. After the development of advanced spotting devices, the feature distance decreased to 60-150 μm (4, 5).

The second type, in-situ synthesized arrays, were first demonstrated using light-directed chemical synthesis, which was initially used by Fodor, the founder of Affymetrix, to synthesize peptide arrays in 1991 (6) and then applied to DNA arrays by Pease et al. in 1994 (7). The light-guided DNA synthesis uses photolithography, a light-targeted removal of a photolabile protecting group at specific array positions, allowing four nucleotides to selectively add to the deprotected DNAs. The light-guide chemistry offers high spatial resolution, so these arrays can achieve a high feature density, e.g., 135,000 per slide with a feature size of 35×35 μm (8). More recently, the feature size was further decreased to 5 μm (9). Major limitations of the in-situ synthesis include the necessity of using many photolithographic masks to control multiple synthesis cycles, making the process slow and costly, and a high synthesis error rate, limiting the maximum DNA length to −100 bp. Other in-situ synthesis methods were also developed, for example, the spotting method using inkjet-style printing to deliver nucleotide building blocks to specific array positions (10). The inkjet printing is compatible with a classical DNA synthesis chemistry based on chemical removal of a DNA protection group, showing an error rate lower than that of the light-directed synthesis, so the DNA length can increase to 200 bp or above. A commercial example from this type is Agilent oligonucleotide microarrays.

Self-assembled arrays come in a variety of feature forms, for example, DNA-coated beads (11-14), DNA nanoballs (15), and polymerase colonies (known as polonies (16, 17) or DNA clusters (18)). Although they are manufactured with distinct methods, they share a common aspect: features are randomly distributed on non-patterned (or random) or patterned arrays and DNA sequence on each feature needs to be determined by a decoding step at a later stage. Walt et al. created random bead arrays on the end of a fiber optic array in which the ends of the fibers were etched to provide a well that is slightly larger than one bead. These beads were optically encoded with different fluorophore combinations to be decoded by fiber-optic fluorescence imaging to determine which oligo was in which position on the array (11, 19-21). This technology was licensed to Illumina and later improved to make larger arrays with more beads. The optical decoding by fluorescent bead labeling limits the total number of unique beads to be distinguished. Thus, a new decoding method involved hybridizing a set of fluorescently labeled oligo probes to DNAs on beads in a pitted glass in a sequential series of steps like DNA sequencing (12). More recently, much more complex self-assembled arrays than bead arrays were generated by next-generation sequencing platforms. Complete Genomics used rolling circle amplification (22) to amplify single-molecule templates to DNA nanoballs of ~200 nanometer in a tube and then immobilize them on a silicon chip for massively parallel DNA sequencing to determine the sequence in each feature (15). Similarly, the most widely implemented Illumina next-generation sequencing used in-gel bridge amplification to in situ amplify single-molecule templates randomly seeded in a linear polyacrylamide coating on glass flowcell surfaces to clonal DNA clusters of ~1 μm for sequencing (18). Due to the small feature sizes, these DNA arrays have ultrahigh feature densities (e.g., >1 million/mm²) and the complicated decoding is solved by sequencing. However, the array fabrication requires sequencing each array, which yet is a costly and slow process. Additionally, the fluorescence imaging used by DNA nanoball and cluster sequencing often partially damage DNAs on the arrays. Thus, these ultradense DNA arrays have not been commercially fabricated for standard DNA array applications. Most recently, the advancement of spatial transcriptomics for mapping biological tissues (23) inspired the use of the high-resolution arrays to achieve cellular and subcellular resolutions (13, 14, 24-28).

DNA arrays with each feature bearing a unique spatial barcode, termed spatially barcoded arrays, are increasingly used for in situ capture and sequencing of RNAs and proteins to map the structure and function of heterogeneous tissues (13, 14, 23-27). To achieve single-cell resolution, DNA arrays require features significantly smaller than cells to delineate different shapes, for example, a feature diameter of low- to sub-micrometer. Traditional spotting (1, 2) or light-directed (6) methods for the deposition or in situ synthesis of sequence-defined oligonucleotides at specific array positions on a substrate often generate features larger than mammalian cells (>10 μm) with significant gaps. Recent advances of spatial transcriptomics used random arrays of smaller features such as DNA-coated beads (13, 14), DNA nanoballs (26), and polonies (28) (or DNA clusters (25)) requiring decoding spatial barcodes by sequencing each array with specialized flowcells or devices (table 2). The ability to print the high-resolution arrays on custom substrates such as microscope slides without individually sequencing them would improve the accessibility, scalability, and flexibility of array-based assays. A possible method for large-scale fabrication is microcontact printing (29) using an elastomeric stamp to simultaneously copy arrayed molecules to a substrate. However, an unsolved problem is to construct a spatially barcoded array on a stamp allowing consecutive printing without progressive decline of the resolution and amounts of copied DNAs.

### SUMMARY

Embodiments of the present disclosure are based in part on the discovery that polonies formed on the surface of an elastomeric, crosslinked polyacrylamide “stamp gel” as templates can be efficiently copied to many other “copy gels” by DNA polymerase-catalyzed chain extension. The gel-to-gel replication described herein reliably achieved submicrometer resolution because all primers and templates were covalently attached to the gels to prevent DNA diffusion. Unlike the traditional stamping requiring “re-inking” the stamp for consecutive printing (See, e.g., S. A. Lange, V. Benes, D. P. Kern, J. K. Horber, A. Bernard, Microcontact printing of DNA molecules. Anal. Chem. 76, 1641-1647 (2004)), enzymatic DNA copying of the disclosure did not consume templates on the stamp. Notably, the stamping was also facilitated by the bridge amplification (L. Gu et al., Multiplex single-molecule interaction profiling of DNA-barcoded proteins. Nature 515, 554-557 (2014); D. R. Bentley et al., Accurate whole human genome sequencing using reversible terminator chemistry. Nature 456, 53-59 (2008)) on gel surfaces to achieve a high copying efficiency and intensify faint prints. To obtain a spatial barcode map for a series of prints, only one or a few copy gels need to be sequenced. Additionally, copy gels could serve as stamps for next fabrication rounds. Polony gels showed an even, continuous feature distribution, restrained the template diffusion in tissue sections, and achieved a high RNA capture efficiency (e.g., a mean of ~1,000 unique molecular identifiers (UMIs)/10×10 μm² in mouse tissue). Gel-based polony-indexed library-sequencing (Pixel-seq) was achieved for single-cell spatial transcriptomics of the mouse brain tissues.

The spatial resolution of DNA array-based spatial transcriptomic and proteomic assays has been compromised by diffusion of analytes (e.g., RNAs and DNA-tagged affinity molecules) in tissue and on an array. So far, these assays all rely on non-directional migration of tissue analytes prior to being captured by array probes. Although tissue samples can be prepared in thin sections (e.g., ≤10-μm thickness) to facilitate the capturing by proximal array features, the lateral diffusion of analytes imposes a major limitation to the assays requiring single-cell or subcellular resolution. One solution to decrease the diffusion is electroblotting by applying an electric field vertical to the tissue section to drive the directional migration of tissue analytes to the array. However, arrays fabricated with traditional methods or requiring sequencing DNA sequences on the arrays use solid supports (e.g., glass, silicon, and polydimethylsiloxane) incompatible with electroblotting. Although modifications of solid support surfaces might render them electrically conductive, for example, indium tin oxide (ITO)-coating of glass surfaces (website: doi.org/10.1101/2022.01.12.476082), such modifications are expensive to make and can affect DNA immobilization to the solid supports. To address this limitation, the DNA stamping method generates DNA arrays on the surface of hydrogels (e.g., polyacrylamide) allowing the gel-based electrophoretic capture of tissue analytes.

In certain aspects, a method of replicating a template microarray is provided. The method comprises the steps of providing a template microarray comprising a plurality of nucleic acid sequence clusters, contacting the template microarray with a copy microarray in the presence of polymerase and nucleotides, removing the template microarray, and amplifying nucleic acid sequences present on the copy microarray to form a plurality of nucleic acid clusters.

In one aspect, a method of replicating a template microarray is provided. The method includes the steps of providing a template microarray comprising a plurality of nucleic acid features where each comprises a plurality of identical single-stranded DNA sequences that form a random or patterned array distribution, contacting the template microarray with a copy microarray in the presence of polymerase and nucleotides to synthesize complementary DNA sequences on the copy microarray, removing the template microarray, amplifying synthesized complementary DNA sequences on the copy microarray to form a plurality of nucleic acid features with the sequences and distribution substantially identical to those of the template microarray, and repeating the replicating process with the same template microarray and a plurality of copy microarrays to produce a plurality of replicated copy microarrays.

In certain exemplary embodiments, the template microarray comprises a solid support having a top surface and a plurality of nucleic acid features on the top surface of the support.

In certain exemplary embodiments, the solid support is a non-porous substrate or a porous substrate, a rigid substrate or an elastomeric substrate, a single-layered substrate or a multilayered substrate, wherein the solid support allows covalent or non-covalent attachment of nucleic acid sequences to its surface, and/or a substrate with a flat surface without or with chemically or photo etched microwell or nanowell structures, wherein nucleic acid sequences are attached to the flat surface or inner surfaces of microwells or nanowells.

In certain exemplary embodiments, the nucleic acid features in the template microarray are selected from the group consisting of clonal DNA clusters, DNA nanoballs, DNA coated beads, DNA spots deposited or synthesized by spotting methods, and DNA spots synthesized by light-directed synthesis methods.

In certain exemplary embodiments, the plurality of nucleic acid features form a random array or a patterned array.

In certain exemplary embodiments, the synthesis of single-stranded DNA sequences in the template microarray is performed by a method selected from the group consisting of chemical synthesis, enzymatic synthesis, template-dependent synthesis, template-independent synthesis, synthesis of double-stranded DNA sequences followed by cleaving of one or two strands of double-stranded DNAs.

In certain exemplary embodiments, the cleaving is performed using one or more enzymatic reagents optionally selected from the group consisting of uracil-specific excision enzymes, 8-oxoguanine DNA glycosylases, and restriction enzymes, or one or more chemical reagents, optionally selected from the group consisting of oxidizing reagents and reducing reagents.

In certain exemplary embodiments, the oxidizing reagent is selected from periodates and lead tetraacetate and the reducing reagent is selected from phosphines, dithiothreitol, dithioerythritol, and L-glutathione.

In certain exemplary embodiments, the copy microarray comprises an elastomeric solid support having a top surface and a plurality of nucleic acid primer sequences on the top surface.

In certain exemplary embodiments, the elastomeric solid support is a non-porous substrate or a or porous substrate, or a single-layered substrate or a multilayered substrate; wherein the elastomeric solid support allows covalent attachment of nucleic acid primer sequences to its surface, conformal contact between two microarray supports, and solid-phase clonal DNA amplification on the support.

In certain exemplary embodiments, a plurality of nucleic acid primers are mixed and evenly cover the top surface of the elastomeric solid support or cover the top surface of the elastomeric solid support in a patterned distribution.

In certain exemplary embodiments, during the contacting step, a part or substantially all of the single-stranded DNA sequences in each feature in the template microarray hybridize to primer sequences in a copy microarray to serve as templates to synthesize complementary DNA sequences.

In certain exemplary embodiments, before removing the template microarray, synthesized complementary DNA sequences on the copy microarray are dissociated from single-stranded DNA sequences on the template microarray by thermal and/or chemical denaturation.

In certain exemplary embodiments, the amplifying step is the solid-phase clonal DNA amplification that is optionally bridge amplification.

In certain exemplary embodiments, a plurality of contacting steps are performed sequentially by contacting the same template microarray to a plurality of copy microarrays to fabricate a plurality of replicated copy microarrays with nearly identical feature nucleic acid sequences and distributions.

In certain exemplary embodiments, a replicated copy microarray can be used as a template microarray for an additional microarray replication process.

In certain exemplary embodiments, a plurality of contacting steps are performed sequentially by replicating a plurality of different template microarrays to the copy microarray to achieve increased feature density and DNA coverage, and decreased feature size on the copy microarray.

In certain exemplary embodiments, the nucleic acid sequences comprise a single or a plurality of probe sequences for hybridization to complementary sequences in target nucleic acid molecules in a sample.

In certain exemplary embodiments, target nucleic acid molecules remain in sectioned tissues where their original cellular and tissue locations do not change or wherein target nucleic acid molecules are released from homogenized cells or tissues where cellular membranes are broken.

In certain exemplary embodiments, target nucleic acid molecules are endogenous nucleic acids in cells or artificially synthesized nucleic acid tags covalently or non-covalently attached to affinity molecules or incorporated into endogenous nucleic acid sequences.

In certain exemplary embodiments, an affinity molecule is selected from the group consisting of a binding protein, an antibody, an antigen-binding fragment of an antibody, a nanobody, a monobody, and a nucleic acid aptamer. In certain exemplary embodiments, the affinity molecule is used to detect one or any combination of proteins, DNAs, RNAs, small-molecule ligands, protein modifications, DNA modifications, RNA modifications, small-molecule ligand modifications, and molecular complexes present in a sample.

In certain exemplary embodiments, different probe sequences are present in different features such that their sequences are associated with feature positions in a microarray.

In certain exemplary embodiments, the same probe or a plurality of different probes are present in substantially all features in a microarray.

In certain exemplary embodiments, in addition to probe sequences, nucleic acid sequences in a feature comprise a barcoding sequence which is associated with a feature position in a microarray.

In certain exemplary embodiments, the probe and/or the barcoding sequences are known or unknown during microarray construction, and can be later determined by DNA sequencing or hybridization methods.

In certain exemplary embodiments, an internal probe sequence in DNAs is exposed at the 3′ end by nucleic acid sequence-specific cleavage methods including, but not limited to, restriction enzyme digestion.

In certain exemplary embodiments, nucleic acid sequences without any probe sequence are added with a single or a plurality of probe sequences by enzymatic methods that are optionally selected from DNA ligation or primer extension after the microarray replication.

In certain exemplary embodiments, feature probe and/or barcoding sequences in microarrays replicated from the same template microarray are substantially identical and are determined by feature sequences in the template microarray or by only sequencing in one or a few out of many copy microarrays.

In certain exemplary embodiments, the template and copy microarrays are used to analyze gene expression and genetic variations of single nucleotide polymorphisms in a sample.

In certain exemplary embodiments, the template and copy microarrays are used to analyze the binding of transcription factors to probe sequences.

In certain exemplary embodiments, a microarray is used to spatially barcode nucleic acids, proteins, and/or small-molecule ligands in a tissue placed onto the microarray by incorporating barcoding sequences into in situ synthesized complementary DNAs to endogenous nucleic acids or artificially synthesized nucleic acid tags.

In certain exemplary embodiments, a spatially barcoded microarray is used to analyze molecular interactions between proteins, nucleic acids, and/or small-molecule ligands in a tissue or homogeneous mixture sample.

In certain exemplary embodiments, the microarray replication is used to amplify natural or synthetic DNAs and/or genes on a chip to minimize bulk polymerase chain reaction (PCR)-induced distortions.

In certain exemplary embodiments, nucleic acid templates and primers are covalently attached to the template and copy microarrays, restraining DNA diffusion to achieve sub-micrometer resolution feature replication.

In certain exemplary embodiments, nucleic acid templates covalently attached to a template microarray have a minimal loss in each contact step, so the template microarray can be repeatedly used for many replication cycles.

In another aspect, a method of replicating a template microarray is provided. The method includes the steps of providing a template microarray comprising a plurality of clonal DNA clusters where each comprises a plurality identical single-stranded DNA sequences that form a random or patterned array distribution, contacting the template microarray with a copy microarray in the presence of DNA polymerase and nucleotides to synthesize complementary DNA sequences on the copy microarray, removing the template microarray, amplifying synthesized complementary DNA sequences on the copy microarray to form a plurality of DNA clusters with the sequences and distribution substantially identical to those in the template microarray, and repeating the replicating process with the template microarray and a plurality of copy microarrays to produce a plurality of replicated copy microarrays.

In certain exemplary embodiments, wherein the template microarray comprises a gel or a substrate with chemically or photo etched microwell or nanowell structures and the copy microarray each comprises a gel.

In certain exemplary embodiments, the gel is selected from the group consisting of a polyacrylamide gel, a hydrogel, and a polydimethylsiloxane gel.

In certain exemplary embodiments, clonal DNA clusters in the template and copy microarrays are amplified by bridge amplification with different cycle numbers to control DNA density or copy number in clusters and cluster sizes.

In another aspect, a method of replicating a template microarray is provide. The method includes the steps of providing a template microarray comprising a polyacrylamide gel having a plurality of clonal DNA clusters attached thereto, contacting the template microarray with a copy microarray comprising a polyacrylamide gel having a plurality of primer sequences attached thereto in the presence of DNA polymerase and nucleotides, removing the template microarray, amplifying nucleic acid sequences present on the copy microarray to form a plurality of clonal DNA clusters with the sequences and distribution substantially identical to those in the template microarray, and repeating the replicating process with the template microarray and a plurality of copy microarrays to produce a plurality of replicated copy microarrays.

In certain exemplary embodiments, the polyacrylamide gel comprises single-layered or multilayered linear polyacrylamide and/or the polyacrylamide crosslinked with one or a plurality of crosslinkers that is optionally selected from the group consisting of N,N′-methylene-bis-acrylamide, N,N′-cystamine-bis-acrylamide, and N,N′-diallyltartardiamide.

In another aspect, a method of generating a replicated copy microarray on a gel suitable for electroblotting assays for tissue or homogeneous mixture samples is provided. The method includes the steps of replicating a template microarray by the method of claim 1 to a copy microarray on the top surface of a porous gel with the top and bottom surfaces exposed a conducting medium, allowing ions to migrate in or out, attaching a tissue section or homogeneous mixture sample to the microarray, applying an electric field vertical to the gel top surface to drive migration of analytes in the sample to the top surface of the gel, and capturing nucleic acid targets on the gel surface by hybridization to probe sequences in the microarray.

In certain exemplary embodiments, a sample is a fresh frozen or formalin-fixed paraffin-embedded (FFPE) tissue section or a homogeneous mixture of one or any combination of nucleic acids, proteins, small-molecule ligands, and DNA-tagged affinity molecules.

In certain exemplary embodiments, the transferring of nucleic acid targets is implemented with a semi-dry or dry electroblotting device.

In another aspect, a method of region-selective analysis of nucleic acid targets captured on one or a plurality of regions of a microarray is provided comprising: replicating a template microarray to a copy microarray having photocleavable probe sequences by a method comprising the steps of providing a template microarray comprising a plurality of nucleic acid sequence clusters, contacting the template microarray with a copy microarray in the presence of polymerase and nucleotides, removing the template microarray, and amplifying nucleic acid sequences present on the copy microarray to form a plurality of nucleic acid clusters; attaching a tissue section or homogeneous mixture sample to the copy microarray; capturing nucleic acid targets on the gel surface by hybridization to probe sequences on the microarray; synthesizing complementary DNA sequences using probe sequences as primers on the microarray; using light to photocleave probes in one or a plurality of microarray regions to selectively release synthesized complementary DNA sequences; and eluting released complementary DNAs, wherein the eluted released complementary DNAs are optionally used for DNA amplification, sequencing, and/or other assays.

In certain exemplary embodiments, probe sequences are linked to a microarray via one or a plurality of photocleavable spacers including, but not limited to, 1-(2-nitrophenyl)ethyl esters.

In another aspect, a method of the region-selective analysis of nucleic acid targets captured on one or a plurality of regions of a microarray is provided comprising: replicating a template microarray to a copy microarray on a gel substrate by a method comprising the steps of providing a template microarray comprising a plurality of nucleic acid sequence clusters, contacting the template microarray with a copy microarray in the presence of polymerase and nucleotides, removing the template microarray, and amplifying nucleic acid sequences present on the copy microarray to form a plurality of nucleic acid clusters; attaching a tissue section or homogeneous mixture sample to the copy microarray; capturing nucleic acid targets on the gel surface by hybridization to probe sequences on the microarray; synthesizing complementary DNA sequences using probe sequences as primers on the microarray; using a micro cutter device to mechanically cut one or a plurality of gel regions and transfer them to a receptable that is optionally a tube; and analyzing complementary DNAs from cut gel pieces by DNA amplification, sequencing, and/or other assays.

In certain exemplary embodiments, the gel substrate is a non-rigid material suitable for mechanical cutting including, but not limited to, a polyacrylamide gel.

In certain exemplary embodiments, the micro cutter device has a microdissection head for cutting small gel regions, a tubing for applying vacuum and pressure to the microdissection head to transfer cut gel pieces to a tube, and an XYZ stage for sampling different gel regions.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 schematically depicts the amplifiable DNA stamping method. Linearized single-stranded polony DNAs are copied from a stamp to many copy gels and copied DNAs are amplified by the bridge amplification to complete the gel replication. A few copy gels are used in the contacting step for the next fabrication rounds or for polony sequencing to create a spatial barcode map; the majority are used for assays.

FIG. 2A and FIG. 2B depict the gel-to-gel DNA copying process (FIG. 2A) automated with a stamping device (FIG. 2B). The device comprises a desktop robotic arm that sequentially places the stamp in four positions in each stamping cycle: A) formamide at 60° C.; B) a stamping buffer at 60° C.; C) a stamping mix including Taq DNA polymerase and dNTPs at 4° C.; and D) a specified position on a copy gel at 95 to 60° C. The stamping pressure is monitored by an electronic balance.

FIG. 3A depicts millimeter-scale images of SYBR Green-stained DNAs in a stamp and a copy gel. To compare the DNA patterns on the gels, templates were seeded on the masked, 40 μm-thick stamp gel and amplified to polonies showing a pattern of the word “Pixel.”

FIG. 3B depicts submicrometer-resolution images of SYBR Green-stained polonies in the copy gels from the 2^{nd}, 10^{th}, and 50^{th} stamping cycles.

FIG. 4 depicts the continuous feature distribution of polony gels. 3D intensity profiles of SYBR Green-stained, discrete, and continuous polonies amplified from templates seeded at the same density by 35 cycles (left). A merged four-color sequencing image (middle) was converted to a spatial barcode map by the pixel-level base calling (right).

FIG. 5A depicts a box plot of the percentages of polonies in copy gels matched the consensus. Data represent mean values of six sampled gel positions each found with 195 to 332 polonies; error bars, standard deviation.

FIG. 5B depicts 2D and 1D density plots of relative positions of polony centers in three copy gels from the consensus. The inner and outer dash circles denote the distances of 0.5 and 1 μm, respectively. n=4,521.

FIG. 5C depicts a box plot of barcode error rates measured by the Illumina sequencing of barcoded DNAs eluted from two copy gels to compare detected barcodes.

FIG. 5D depicts a comparison of polony amplification efficiencies for different gel substrates. The linear polyacrylamide substrate was prepared by a reported Illumina method. Left four clusters: crosslinked polyacrylamide; right-most cluster, linear polyacrylamide.

FIG. 5E depicts a violin plot of measured diameters of polonies at different densities. N=about 0.6 to 1 million.

FIG. 6 depicts the principle of Pixel-seq. A polony gel captures RNAs from the gel-contacting layer in a cryosectioned tissue. Spatially barcoded cDNAs are synthesized and introduced with a 3′ universal sequence by a template-switching oligo to facilitate cDNA amplification. They are sequenced to associate RNAs to their gel locations to create a transcript map. A k-nearest neighbor network is built on the map where each barcode represented a node. Edge weights are calculated as a function of the UMI counts, the distance and the transcript similarity between two connected barcodes. The weighted network is segmented by a graph-based algorithm to create cell masks to aggregate transcripts for single-cell data analyses.

FIG. 7 depicts gel capturing of RNAs from a single cell layer in a tissue section. Confocal image analysis of nuclei in a mouse OB section atop a polony gel and the cDNAs synthesized on the gel. Two found layers of the nuclei that are proximal (0 μm) and distal (6 μm) to the gel surface are overlaid with cDNA signals.

FIG. 8A depicts a representative UMI density map of a coronal OB section. UMI densities were measured on each image pixel (0.325×0.325 μm²).

FIG. 8B and FIG. 8C depicts a comparison of RNA capture efficiencies of Pixel-seq and other spatial transcriptomics methods. Most recent OB or other available datasets were used. Pixel-seq data were counted on bins of 7×7 (2 μm, FIG. 8B) and 33×33 (10 μm, FIG. 8C) pixels. Feature gaps of the arrays used by other methods were not considered in this comparison.

FIG. 9A depicts a comparison of polony patterns at equivalent locations of three copy gels stamped under the pressures of about 1×10⁴, about 2×10⁴, and about 3×10⁴ Pa. Specifically, the forces equivalent to the weights of about 50, about 100, and about 150 grams were applied to a stamp of about 7×7 mm². The low-density polonies were amplified to facilitate the comparison of feature patterns.

FIG. 9B depicts a box plot of the percentages of polonies in copy gels matched in the consensus. Data represents mean values of six sampled gel positions each found with 127 to 236 polonies; error bars, standard deviation.

FIG. 10A and FIG. 10B depict representative images of polonies amplified in a crosslinked (FIG. 10A) and a linear (FIG. 10B) polyacrylamide (PAA) gel. The linear PAA gel was fabricated following the reported Illumina method. DNA templates were seeded at the same density on the gels and amplified with the indicated cycle numbers before DNA staining with SYBR Green. All images were acquired with the same imaging setting. The background-subtracted fluorescence signals are compared in FIG. 5D.

FIG. 11 depicts four-color sequencing images for comparing polony sizes at increased densities. Templates were seeded at specific densities and amplified by 35 cycles. Due to the polony exclusion effect, even at higher feature densities than were typically used (e.g., about 1.2 million/mm² with an average feature size of about 0.5 μm), most polonies in different colors show clear boundaries, suggesting that they can be sequenced with a high-resolution imaging setup. Some dark regions in the images were also occupied by polonies with no sequencing signals because some randomly generated barcodes had the TaqI cleavage site(s).

FIG. 12A depicts signals of Cy5-labeling of cDNAs synthesized from the gel-captured RNAs delineate shapes of cell bodies. A 10-μm mouse olfactory bulb-isocortex section was assayed. AON, anterior olfactory nucleus; AOB, accessory olfactory bulb; MOB, main olfactory bulb; CTX, cerebral cortex.

FIG. 12B depicts the quantification of the lateral template diffusion. Comparison of Cy5-labeling of cDNA signals to a reference, the stained nuclei imaged immediately after placing a 10-μm cryosectioned mouse OB section on a dry polony gel pre-soaked with SYTOX Green. Images were acquired with an epifluorescence microscopy used for polony gel sequencing. In the zoom-in images, nuclei are significantly more than cDNA signals because the former were from multiple cell layers in the section, but the latter were only from a gel-contacting single cell layer.

FIG. 12C depicts the quantification of the lateral template diffusion. The scaling factor was calculated for the images in FIG. 12B. Data represent ten regions (250×250 pixels, 1 pixel=0.65 μm) sampled from (B); error bars, standard error of the mean. The scaling factor of 1.028±0.016 is equivalent to a diffusion distance of 0.04 to 0.4 μm.

FIG. 13 depicts an illustration of capturing RNAs from a single cell layer during the gel wetting.

FIG. 14 depicts a flowchart of the Pixel-seq assay to construct a cDNA sequencing library.

FIG. 15 depicts a template sequence (SEQ ID NO: 1) used for polony construction.

FIG. 16 depicts copying of DNA clusters from an Illumina NovaSeq flowcell to a copy gel. A matched concentric mark (with no DNA clusters) is found in the copy gel.

FIG. 17 depicts an electrophoretic capture of tissue analytes on the top surface of a gel-based DNA microarray. The gel has small pore sizes to prevent the analytes from migrating into the gel.

FIG. 18 depicts light-guided region-specific release of nucleic acid targets captured on selected DNA array regions.

FIG. 19 depicts the mechanical microdissection of selected gel regions for region-specific analysis of nucleic acid targets captured on a DNA array.

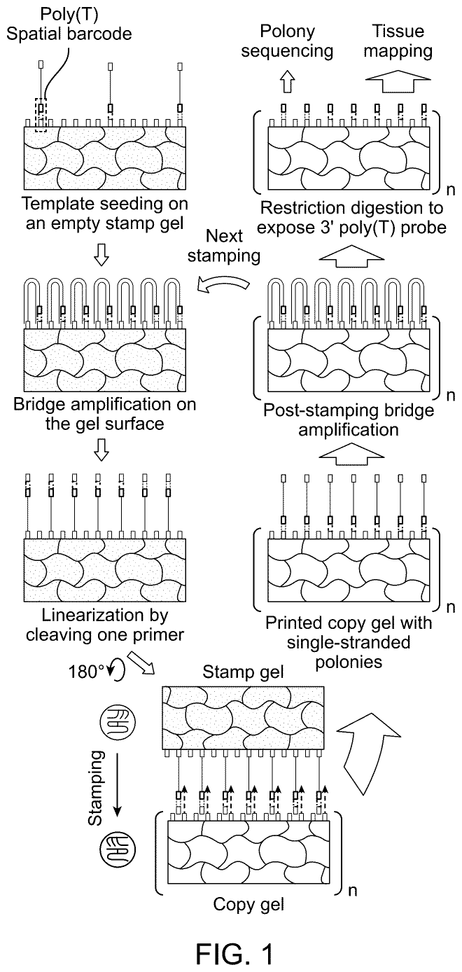

### DETAILED DESCRIPTION

Before the disclosure is described, it is to be understood that disclosure is not limited to particular methods and experimental conditions described, as such methods and conditions may vary. It is also to be understood that the terminology used herein is for the purpose of describing particular embodiments only, and is not intended to be limiting, because the scope of the disclosure will be limited only by the appended claims.

Unless defined otherwise, all technical and scientific terms used herein have the same meaning as commonly understood by one of ordinary skill in the art to which this disclosure belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

In various embodiments, the methods disclosed herein comprise amplification of nucleic acids including, for example, polynucleotides, oligonucleotides and/or oligonucleotide fragments. Amplification methods may comprise contacting a nucleic acid sequence with one or more primers (e.g., primers that are complementary to barcode sequences) that specifically hybridize to the nucleic acid under conditions that facilitate hybridization and chain extension. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self-sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, isothermal amplification (e.g., isothermal bridge amplification (IBA), rolling circle amplification (RCA), hyperbranched rolling circle amplification (HRCA), strand displacement amplification (SDA), helicase-dependent amplification (HDA), PWGA or any other nucleic acid amplification method using techniques well known to those of skill in the art.

“Polymerase chain reaction,” or “PCR,” refers to a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Typically, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature greater than 90° C., primers annealed at a temperature in the range 50-75° C., and primers extended at a temperature in the range 72-78° C. In certain embodiments, a double stranded target nucleic acid may be denatured at a temperature greater than 90° C. in a conventional PCR using Taq DNA polymerase, or by adding formamide at 60° C. in isothermal bridge amplification using Bst polymerase.

The term “PCR” encompasses derivative forms of the reaction, including but not limited to, RT-PCR, real-time PCR, nested PCR, quantitative PCR, multiplexed PCR, assembly PCR and the like. Reaction volumes range from a few hundred nanoliters, e.g., 200 nL, to a few hundred microliters, e.g., 200 microliters. “Reverse transcription PCR,” or “RT-PCR,” means a PCR that is preceded by a reverse transcription reaction that converts a target RNA to a complementary single stranded DNA, which is then amplified, e.g., Tecott et al., U.S. Pat. No. 5,168,038. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al., U.S. Pat. No. 5,210,015 (“Tagman”); Wittwer et al., U.S. Pat. Nos. 6,174,670 and 6,569,627 (intercalating dyes); Tyagi et al., U.S. Pat. No. 5,925,517 (molecular beacons). Detection chemistries for real-time PCR are reviewed in Mackay et al., Nucleic Acids Research, 30:1292-1305 (2002). “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al. (1999) Anal. Biochem., 273:221-228 (two-color real-time PCR). Usually, distinct sets of primers are employed for each sequence being amplified. “Quantitative PCR” means a PCR designed to measure the abundance of one or more specific target sequences in a sample or specimen. Techniques for quantitative PCR are well-known to those of ordinary skill in the art, as exemplified in the following references: Freeman et al., Biotechniques, 26:112-126 (1999); Becker-Andre et al., Nucleic Acids Research, 17:9437-9447 (1989); Zimmerman et al., Biotechniques, 21:268-279 (1996); Diviacco et al., Gene, 122:3013-3020 (1992); Becker-Andre et al., Nucleic Acids Research, 17:9437-9446 (1989); and the like.

In certain embodiments, methods of determining the sequence of one or more nucleic acid sequences of interest, e.g., polynucleotides, oligonucleotides and/or oligonucleotide fragments, are provided. Determination of the sequence of a nucleic acid sequence of interest can be performed using variety of sequencing methods known in the art including, but not limited to, sequencing by hybridization (SBH), sequencing by ligation (SBL), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (polony) sequencing (U.S. Pat. Nos. 6,432,360, 6,485,944 and 6,511,803, and PCT/US05/06425); nanogrid rolling circle sequencing (rolony) (U.S. Ser. No. 12/120,541, filed May 14, 2008), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., on cyclic array sequencing using platforms such as Roche 454, Illumina Solexa, AB-SOLiD, Helicos, Polonator platforms and the like, can also be utilized. High-throughput sequencing methods are described in U.S. Ser. No. 61/162,913, filed Mar. 24, 2009. A variety of light-based sequencing technologies are known in the art (Landegren et al. (1998) Genome Res. 8:769-76; Kwok (2000) Pharmocogenomics 1:95-100; and Shi (2001) Clin. Chem. 47:164-172).

Embodiments of the present invention are directed to polynucleotides, oligonucleotides, small molecules, substrates, test compounds and the like having one or two or more labels (e.g., barcode sequences) attached thereto. As used herein, the term “barcode” refers to a unique oligonucleotide sequence that allows a corresponding nucleic acid sequence (e.g., an oligonucleotide fragment) to be identified, retrieved and/or amplified. In certain embodiments, barcodes can each have a length within a range of from 4 to 36 nucleotides, or from 6 to 30 nucleotides, or from 8 to 20 nucleotides. In certain exemplary embodiments, a barcode has a length of 4 nucleotides. In certain aspects, the melting temperatures of barcodes within a set are within 10° C. of one another, within 5° C. of one another, or within 2° C. of one another. In other aspects, barcodes are members of a minimally cross-hybridizing set. That is, the nucleotide sequence of each member of such a set is sufficiently different from that of every other member of the set that no member can form a stable duplex with the complement of any other member under stringent hybridization conditions. In one embodiment, the nucleotide sequence of each member of a minimally cross-hybridizing set differs from those of every other member by at least two nucleotides. Barcode technologies are known in the art and are described in Winzeler et al. (1999) Science 285:901; Brenner (2000) Genome Biol. 1:1 Kumar et al. (2001) Nature Rev. 2:302; Giaever et al. (2004) Proc. Natl. Acad. Sci. USA 101:793; Eason et al. (2004) Proc. Natl. Acad. Sci. USA 101:11046; and Brenner (2004) Genome Biol. 5:240.

In certain embodiments, one or more markers are used to detect and/or retrieve (i.e. purify) polynucleotides, oligonucleotides, small molecules, substrates, test compounds and the like described herein. Examples of detectable and/or retrievable markers include various radioactive moieties, enzymes, prosthetic groups, fluorescent markers, luminescent markers, bioluminescent markers, metal particles, protein-protein binding pairs, protein-antibody binding pairs and the like. Detectable markers are commercially available from a variety of sources.

In certain aspects of the invention, detectable and/or retrievable proteins and/or protein tags are provided. Examples of detectable fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like. Examples of detectable bioluminescent proteins include, but are not limited to, luciferase (e.g., bacterial, firefly, click beetle and the like), luciferin, aequorin and the like. Examples of detectable and/or retrievable enzyme systems include, but are not limited to, galactosidases, glucorinidases, phosphatases, peroxidases, cholinesterases and the like.

Biotin, or a derivative thereof, may also be used as a detectable and/or retrievable label, and subsequently bound by a detectably labeled avidin/streptavidin derivative (e.g. phycoerythrin-conjugated streptavidin), or a labeled anti-biotin antibody. Digoxigenin may be expressed subsequently bound by a labeled anti-digoxigenin antibody (e.g. fluoresceinated anti-digoxigenin). In general, any member of a conjugate pair may be incorporated into a detection oligonucleotide provided that a detectably labeled conjugate partner can be bound to permit detection. As used herein, the term antibody refers to an antibody molecule of any class, or any sub-fragment thereof, such as a Fab.

Other suitable labels for detection and/or retrieval include one or more protein tags. As used herein, the term “protein tag” refers to a heterologous polypeptide sequence linked to a polymerase of the invention. Protein tags include, but are not limited to, Avi tag (GLNDIFEAQKIEWHE) (SEQ ID NO: 2), calmodulin tag (KRRWKKNFIAVSAANRFKKISSSGAL) (SEQ ID NO: 3), FLAG tag (DYKDDDDK) (SEQ ID NO: 4), HA tag (YPYDVPDYA) (SEQ ID NO: 5), His tag (HHHHHH) (SEQ ID NO: 6), Myc tag (EQKLISEEDL) (SEQ ID NO: 7), S tag (KETAAAKFERQHMDS) (SEQ ID NO: 8), SBP tag (MDEKTTGWRGGHVVEGLAGELEQLRARLEHHPQ GQREP) (SEQ ID NO: 9), Softag 1 (SLAELLNAGLGGS) (SEQ ID NO: 10), Softag 3 (TQDPSRVG) (SEQ ID NO: 11), V5 tag (GKPIPNPLLGLDST) (SEQ ID NO: 12), Xpress tag (DLYDDDDK) (SEQ ID NO: 13), Isopeptag (TDKDMTITFTNKKDAE) (SEQ ID NO: 14), SpyTag (AHIVMVDAYKPTK) (SEQ ID NO: 15), streptactin tag (Strep-tag II: WSHPQFEK) (SEQ ID NO: 16) and the like.

Detection and/or retrieval method(s) used will depend on the particular detectable labels used in the microorganism. In certain exemplary embodiments, microorganisms may be selected for, screened for and/or retrieved using a microscope, a spectrophotometer, a tube luminometer or plate luminometer, x-ray film, magnetic fields, a scintillator, a fluorescence activated cell sorting (FACS) apparatus, a chromatography apparatus, a microfluidics apparatus, a bead-based apparatus or the like.

Terms and symbols of nucleic acid chemistry, biochemistry, genetics, and molecular biology used herein follow those of standard treatises and texts in the field, e.g., Komberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, or from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See Kanehisa (1984) Nucl. Acids Res. 12:203.

“Substantially identical” refers to two or more sequences (e.g., DNA sequences) or two or more microarrays (e.g., template microarrays and/or copy microarrays) that are about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% identical to each other.

“Complex” refers to an assemblage or aggregate of molecules in direct or indirect contact with one another. In one aspect, “contact,” or more particularly, “direct contact,” in reference to a complex of molecules or in reference to specificity or specific binding, means two or more molecules are close enough so that attractive noncovalent interactions, such as van der Waal forces, hydrogen bonding, ionic and hydrophobic interactions, and the like, dominate the interaction of the molecules. In such an aspect, a complex of molecules is stable in that under assay conditions the complex is thermodynamically more favorable than a non-aggregated, or non-complexed, state of its component molecules. As used herein, “complex” refers to a duplex or triplex of polynucleotides or a stable aggregate of two or more proteins. In regard to the latter, a complex is formed by an antibody specifically binding to its corresponding antigen.

“Duplex” refers to at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one aspect, stable duplex means that a duplex structure is not destroyed by a stringent wash, e.g., conditions including temperature of about 5° C. less that the Tm of a strand of the duplex and low monovalent salt concentration, e.g., less than 0.2 M, or less than 0.1 M. “Perfectly matched” in reference to a duplex means that the polynucleotide or oligonucleotide strands making up the duplex form a double stranded structure with one another such that every nucleotide in each strand undergoes Watson-Crick base pairing with a nucleotide in the other strand. The term “duplex” comprehends the pairing of nucleoside analogs, such as deoxyinosine, nucleosides with 2-aminopurine bases, PNAs, and the like, which may be employed. A “mismatch” in a duplex between two oligonucleotides or polynucleotides means that a pair of nucleotides in the duplex fails to undergo Watson-Crick bonding.

“Genetic locus,” or “locus” refers to a contiguous sub-region or segment of a genome. As used herein, genetic locus, or locus, may refer to the position of a nucleotide, a gene, or a portion of a gene in a genome, including mitochondrial DNA, or it may refer to any contiguous portion of genomic sequence whether or not it is within, or associated with, a gene. In one aspect, a genetic locus refers to any portion of genomic sequence, including mitochondrial DNA, from a single nucleotide to a segment of few hundred nucleotides, e.g. 100-300, in length. Usually, a particular genetic locus may be identified by its nucleotide sequence, or the nucleotide sequence, or sequences, of one or both adjacent or flanking regions. In another aspect, a genetic locus refers to the expressed nucleic acid product of a gene, such as an RNA molecule or a cDNA copy thereof.

“Hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tm for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1st Ed., BIOS Scientific Publishers Limited (1999). “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

“Kit” refers to any delivery system for delivering materials or reagents for carrying out a method of the invention. In the context of assays, such delivery systems include systems that allow for the storage, transport, or delivery of reaction reagents (e.g., primers, enzymes, microarrays, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials for assays of the invention. Such contents may be delivered to the intended recipient together or separately. For example, a first container may contain an enzyme for use in an assay, while a second container contains primers.

“Ligation” means to form a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references: Whitely et al., U.S. Pat. No. 4,883,750; Letsinger et al., U.S. Pat. No. 5,476,930; Fung et al., U.S. Pat. No. 5,593,826; Kool, U.S. Pat. No. 5,426,180; Landegren et al., U.S. Pat. No. 5,871,921; Xu and Kool (1999) Nucl. Acids Res. 27:875; Higgins et al., Meth. in Enzymol. (1979) 68:50; Engler et al. (1982) The Enzymes, 15:3 (1982); and Namsaraev, U.S. Patent Pub. 2004/0110213.

“Amplifying” includes the production of copies of a nucleic acid molecule of the array or a nucleic acid molecule bound to a bead via repeated rounds of primed enzymatic synthesis. “In situ” amplification indicated that the amplification takes place with the template nucleic acid molecule positioned on a support or a bead, rather than in solution. In situ amplification methods are described in U.S. Pat. No. 6,432,360.

“Support” can refer to a solid substrate that is insoluble in aqueous liquid and upon which nucleic acid molecules of a nucleic acid array are placed. The support can be non-porous or porous, rigid or elastomeric, and single-layered or multilayered. “Elastomeric solid support” refers to a soft support that can adjust its shape under a pressure to achieve conformal contact between its surface and another solid surface, and then regain its original shape when the pressure is removed from the support. Exemplary non-porous supports include, but are not limited to, glass, silicon, and polydimethylpolysiloxane. Exemplary porous supports include, but are not limited to, hydrogels such as linear and cross-linked polyacrylamide gels. Exemplary rigid supports include, but are not limited to, glass and silicon. Exemplary elastomeric supports include, but are not limited to, polydimethylpolysiloxane, elastic gels and hydrogels. The support can be solid or semi-solid. “Semi-solid” refers to a compressible matrix with both a solid and a liquid component, wherein the liquid occupies pores, spaces or other interstices between the solid matrix elements. Semi-solid supports can be selected from polyacrylamide, cellulose, polyamide (nylon) and crossed linked agarose, dextran and polyethylene glycol.

“Randomly-patterned” or “random” refers to non-ordered, non-Cartesian distribution (in other words, not arranged at pre-determined points along the x- or y-axes of a grid or at defined “clock positions,” degrees or radii from the center of a radial pattern) of nucleic acid molecules over a support, which is not achieved through an intentional design (or program by which such design may be achieved) or by placement of individual nucleic acid features. Such a “randomly-patterned” or “random” array of nucleic acids may be achieved by dropping, spraying, plating or spreading a solution, emulsion, aerosol, vapor or dry preparation comprising a pool of nucleic acid molecules onto a support and allowing the nucleic acid molecules to settle onto the support without intervention in any manner to direct them to specific sites thereon. Arrays of the invention can be randomly patterned or random.

“Heterogeneous” refers to a population or collection of nucleic acid molecules that comprises a plurality of different sequences. According to one aspect, a heterogeneous pool of oligonucleotide sequences is provided with an article of manufacture (e.g., a microarray).

“Nucleoside” as used herein includes the natural nucleosides, including 2′-deoxy and 2′-hydroxyl forms, e.g., as described in Komberg and Baker, DNA Replication, 2nd Ed. (Freeman, San Francisco, 1992). “Analogs” in reference to nucleosides includes synthetic nucleosides having modified base moieties and/or modified sugar moieties, e.g., described by Scheit, Nucleotide Analogs (John Wiley, New York, 1980); Uhlman and Peyman, Chemical Reviews, 90:543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al., Exp. Opin. Ther. Patents, 6: 855-870 (1996); Mesmaeker et al., Current Opinion in Structural Biology, 5:343-355 (1995); and the like. Exemplary types of polynucleotides that can enhance duplex stability include oligonucleotide phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

As used herein, the terms “nucleic acid molecule,” “nucleic acid sequence,” “nucleic acid fragment,” “oligonucleotide,” “oligonucleotide fragment” and “polynucleotide” are used interchangeably and are intended to include, but are not limited to, a polymeric form of nucleotides that may have various lengths, either deoxyribonucleotides or ribonucleotides, or analogs thereof. Nucleic acid molecules include single stranded DNA (ssDNA), double stranded DNA (dsDNA), single stranded RNA (ssRNA) and double stranded RNA (dsRNA). Different nucleic acid molecules may have different three-dimensional structures, and may perform various functions, known or unknown. Non-limiting examples of nucleic acid molecules include a gene, a gene fragment, a genomic gap, an exon, an intron, intergenic DNA (including, without limitation, heterochromatic DNA), messenger RNA (mRNA), transfer RNA, ribosomal RNA, ribozymes, small interfering RNA (siRNA), miRNA, small nucleolar RNA (snoRNA), cDNA, recombinant polynucleotides, branched polynucleotides, plasmids, vectors, isolated DNA of a sequence, isolated RNA of a sequence, nucleic acid probes, and primers. Nucleic acid molecules useful in the methods described herein may comprise natural nucleic acid sequences and variants thereof, artificial nucleic acid sequences, or a combination of such sequences.

An oligonucleotide sequence refers to a linear polymer of natural or modified nucleosidic monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. An “oligonucleotide fragment” refers to an oligonucleotide sequence that has been cleaved into two or more smaller oligonucleotide sequences. Oligonucleotides comprising probes or primers usually have lengths in the range of from 12 to 60 nucleotides, and more usually, from 18 to 40 nucleotides. Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like.

Typically, nucleosidic monomers are linked by phosphodiester bonds. Whenever an oligonucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Typically, oligonucleotides comprise the four natural deoxynucleotides; however, they may also comprise ribonucleosides or non-natural nucleotide analogs. It is clear to those skilled in the art when oligonucleotides having natural or non-natural nucleotides may be employed in methods and processes described herein. For example, where processing by an enzyme is called for, usually oligonucleotides consisting solely of natural nucleotides are required. Likewise, where an enzyme has specific oligonucleotide or polynucleotide substrate requirements for activity, e.g., single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al., Molecular Cloning, Second Edition (Cold Spring Harbor Laboratory, New York, 1989), and like references. Oligonucleotides and polynucleotides may be single stranded or double stranded.

Nucleic acid molecules may optionally include one or more non-standard nucleotide(s), nucleotide analog(s) and/or modified nucleotides. Examples of modified nucleotides include, but are not limited to diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone.

In certain exemplary embodiments, large polynucleotides are provided. In certain aspects, isolation techniques that maximize the lengths of polynucleotides (e.g., DNA molecules) obtained are used. For example, in situ lysis or deproteinization (e.g., with EDTA, detergent, protease, any combinations thereof and the like) after agarose embedding (as routinely performed for pulsed field gel electrophoresis) can be used to obtain polynucleotides.

Nucleic acid molecules may be isolated from natural sources or purchased from commercial sources. Oligonucleotide sequences (e.g., barcodes) may also be prepared by any suitable method, e.g., standard phosphoramidite methods such as those described by Beaucage and Carruthers ((1981) Tetrahedron Lett. 22: 1859) or the triester method according to Matteucci et al. (1981) J. Am. Chem. Soc. 103:3185), or by other chemical methods using either a commercial automated oligonucleotide synthesizer or high-throughput, high-density array methods known in the art (see U.S. Pat. Nos. 5,602,244, 5,574,146, 5,554,744, 5,428,148, 5,264,566, 5,141,813, 5,959,463, 4,861,571 and 4,659,774, incorporated herein by reference in its entirety for all purposes). Pre-synthesized oligonucleotides may also be obtained commercially from a variety of vendors.

Nucleic acid molecules may be obtained from one or more biological samples. As used herein, a “biological sample” may be a single cell or many cells. A biological sample may comprise a single cell type or a combination of two or more cell types. A biological sample further includes a collection of cells that perform a similar function such as those found, for example, in a tissue. Accordingly, certain aspects of the invention are directed to biological samples containing one or more tissues. As used herein, a tissue includes, but is not limited to, epithelial tissue (e.g., skin, the lining of glands, bowel, skin and organs such as the liver, lung, kidney), endothelium (e.g., the lining of blood and lymphatic vessels), mesothelium (e.g., the lining of pleural, peritoneal and pericardial spaces), mesenchyme (e.g., cells filling the spaces between the organs, including fat, muscle, bone, cartilage and tendon cells), blood cells (e.g., red and white blood cells), neurons, germ cells (e.g., spermatozoa, oocytes), amniotic fluid cells, placenta, stem cells and the like. A tissue sample includes microscopic samples as well as macroscopic samples.

In certain aspects, nucleic acid sequences derived or obtained from one or more organisms are provided. As used herein, the term “organism” includes, but is not limited to, a human, a non-human primate, a cow, a horse, a sheep, a goat, a pig, a dog, a cat, a rabbit, a mouse, a rat, a gerbil, a frog, a toad, a fish (e.g., Danio rerio) a roundworm (e.g., C. elegans) and any transgenic species thereof. The term “organism” further includes, but is not limited to, a yeast (e.g., S. cerevisiae) cell, a yeast tetrad, a yeast colony, a bacterium, a bacterial colony, a virion, virosome, virus-like particle and/or cultures thereof, and the like.

Isolation, extraction or derivation of nucleic acid sequences may be carried out by any suitable method. Isolating nucleic acid sequences from a biological sample generally includes treating a biological sample in such a manner that nucleic acid sequences present in the sample are extracted and made available for analysis. Any isolation method that results in extracted nucleic acid sequences may be used in the practice of the present invention. It will be understood that the particular method used to extract nucleic acid sequences will depend on the nature of the source.

Methods of DNA extraction are well-known in the art. A classical DNA isolation protocol is based on extraction using organic solvents such as a mixture of phenol and chloroform, followed by precipitation with ethanol (J. Sambrook et al., “Molecular Cloning: A Laboratory Manual,” 1989, 2nd Ed., Cold Spring Harbor Laboratory Press: New York, N.Y.). Other methods include: salting out DNA extraction (P. Sunnucks et al., Genetics, 1996, 144: 747-756; S. M. Aljanabi and I. Martinez, Nucl. Acids Res. 1997, 25: 4692-4693), trimethylammonium bromide salts DNA extraction (S. Gustincich et al., BioTechniques, 1991, 11: 298-302) and guanidinium thiocyanate DNA extraction (J. B. W. Hammond et al., Biochemistry, 1996, 240: 298-300). A variety of kits are commercially available for extracting DNA from biological samples (e.g., BD Biosciences Clontech (Palo Alto, Calif.): Epicentre Technologies (Madison, Wis.); Gentra Systems, Inc. (Minneapolis, Minn.); MicroProbe Corp. (Bothell, Wash.); Organon Teknika (Durham, N.C.); and Qiagen Inc. (Valencia, Calif)).

Methods of RNA extraction are also well-known in the art (see, for example, J. Sambrook et al., “Molecular Cloning: A Laboratory Manual” 1989, 2nd Ed., Cold Spring Harbour Laboratory Press: New York) and several kits for RNA extraction from bodily fluids are commercially available (e.g., Ambion, Inc. (Austin, Tex.); Amersham Biosciences (Piscataway, N.J.); BD Biosciences Clontech (Palo Alto, Calif); BioRad Laboratories (Hercules, Calif); Dynal Biotech Inc. (Lake Success, N.Y.); Epicentre Technologies (Madison, Wis.); Gentra Systems, Inc. (Minneapolis, Minn.); GIBCO BRL (Gaithersburg, Md.); Invitrogen Life Technologies (Carlsbad, Calif); MicroProbe Corp. (Bothell, Wash.); Organon Teknika (Durham, N.C.); Promega, Inc. (Madison, Wis.); and Qiagen Inc. (Valencia, Calif.)).

“Polymorphism” or “genetic variant” means a substitution, inversion, insertion, or deletion of one or more nucleotides at a genetic locus, or a translocation of DNA from one genetic locus to another genetic locus. In one aspect, polymorphism means one of multiple alternative nucleotide sequences that may be present at a genetic locus of an individual and that may comprise a nucleotide substitution, insertion, or deletion with respect to other sequences at the same locus in the same individual, or other individuals within a population. An individual may be homozygous or heterozygous at a genetic locus; that is, an individual may have the same nucleotide sequence in both alleles, or have a different nucleotide sequence in each allele, respectively. In one aspect, insertions or deletions at a genetic locus comprises the addition or the absence of from 1 to 10 nucleotides at such locus, in comparison with the same locus in another individual of a population (or another allele in the same individual). Typically, insertions or deletions are with respect to a major allele at a locus within a population, e.g., an allele present in a population at a frequency of fifty percent or greater.

“Primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Typically, primers are extended by a DNA polymerase. Primers usually have a length in the range of between 3 to 36 nucleotides, also 5 to 24 nucleotides, also from 14 to 36 nucleotides. Primers within the scope of the disclosure include orthogonal primers, amplification primers, constructions primers and the like. Pairs of primers can flank a sequence of interest or a set of sequences of interest. Primers and probes can be degenerate in sequence. Primers within the scope of the present invention bind adjacent to a target sequence (e.g., an oligonucleotide fragment, a barcode sequence or the like).

“Specific” or “specificity” in reference to the binding of one molecule to another molecule, such as an amplification or sequencing primer to a barcode sequence, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. In certain aspects, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak non-covalent chemical interactions, such as van der Waal forces, hydrogen bonding, base-stacking interactions, ionic and hydrophobic interactions, and the like, dominate the interaction of the molecules.

“Spectrally resolvable” in reference to a plurality of fluorescent labels means that the fluorescent emission bands of the labels are sufficiently distinct, i.e., sufficiently non-overlapping, that molecular tags to which the respective labels are attached can be distinguished on the basis of the fluorescent signal generated by the respective labels by standard photodetection systems, e.g., employing a system of band pass filters and photomultiplier tubes, or the like, as exemplified by the systems described in U.S. Pat. Nos. 4,230,558; 4,811,218, or the like, or in Wheeless et al., pgs. 21-76, in Flow Cytometry: Instrumentation and Data Analysis (Academic Press, New York, 1985). In one embodiment, spectrally resolvable organic dyes, such as fluorescein, rhodamine, and the like, means that wavelength emission maxima are spaced at least 20 nm apart, and in another aspect, at least 40 nm apart. In another aspect, chelated lanthanide compounds, quantum dots, and the like, spectrally resolvable means that wavelength emission maxima are spaced at least 10 nm apart, and in a further aspect, at least 15 nm apart.

“Tm” is used in reference to “melting temperature.” Melting temperature is the temperature at which a population of double-stranded nucleic acid molecules becomes half dissociated into single strands. Several equations for calculating the Tm of nucleic acids are well-known in the art. As indicated by standard references, a simple estimate of the Tm value may be calculated by the equation. Tm=81.5+0.41 (% G+C), when a nucleic acid is in aqueous solution at 1 M NaCl (see e.g., Anderson and Young, “Quantitative Filter Hybridization,” in Nucleic Acid Hybridization (1985). Other references (e.g., Allawi, H. T. & Santa Lucia, J., Jr., Biochemistry 36, 10581-94 (1997)) include alternative methods of computation which take structural and environmental, as well as sequence characteristics into account for the calculation of Tm.

The present disclosure is further illustrated by the following example which should not be construed as further limiting. The contents of the figures, tables and all references, patents and published patent applications cited throughout this application are expressly incorporated herein by reference for all purposes.

Furthermore, in accordance with the present disclosure there may be employed conventional molecular biology, microbiology, and recombinant DNA techniques within the skill of the art. Such techniques are explained fully in the literature. See, e.g., Green & Sambrook, Molecular Cloning: A Laboratory Manual, Fourth Edition (2012) Cold Spring Harbor Laboratory Press, Cold Spring Harbor, New York; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXAMPLE

The following example is put forth so as to provide those of ordinary skill in the art with a complete disclosure and description of how to make and use the methods and compositions featured in the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Amplifiable, Submicron-Resolution Replication of DNA Arrays

This example describes a vastly scalable stamping method to fabricate polony gels, arrays of ~1-micrometer clonal DNA clusters each bearing a unique sequence. By coupling enzymatic DNA copying between hydrogel surfaces and bridge amplification of copied DNAs, this method enables the repeatable replication of spatially patterned sequences on the gels with submicrometer resolution. The gel stamping was implemented with a simple robotic arm and off-the-shelf reagents. Compared with the fabrication of similar DNA cluster arrays requiring sequencing polonies in each array, our cost and time decreased by at least 35- and 7-fold, respectively. The amplifiable replication method should be widely appliable to copy and amplify DNAs from many types of DNA arrays to hydrogel surfaces. Polony gels are uniquely suited for array-based spatial transcriptomics and spatial proteomics applications by offering the high resolution and sensitivity for tissue mapping assays. Additionally, they will decrease the cost and increase the throughput and resolution for other DNA microarray-based applications, for example, gene expression analysis, transcription factor binding analysis, and genotyping.

### Experimental Set-Up

### Materials and Methods

### Polony Template Construction

370-base pair (bp) DNA templates bearing a 24-bp random sequence were synthesized by Integrated DNA Technologies (IDT). They were PCR amplified (Taq 2× master mix; New England Biolabs (NEB), M0270) with bridge PCR primers (BA(+) and BA(−); Table 1) for 15 cycles and size selected by 2% agar gel. Purified DNAs were quantified by QUBIT 4 fluorometer (Thermo Fisher Scientific), diluted to 1 nM, and aliquoted for stamp gel preparation or storage at −20′C.

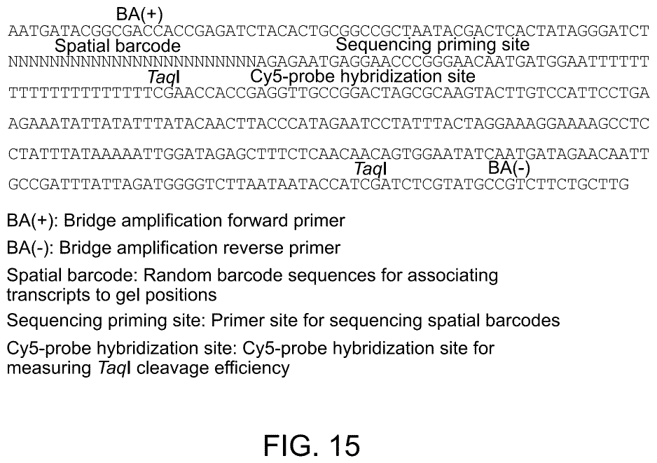

### Template Sequence—See FIG. 15.

TABLE 1
Oligonucleotides used in this study

| Name | Sequence (5′ −> 3′) |
|---|---|
| PS-BA(+) | /5PS/-TTTTTTTTTTAATGATACGGCGACCACCGAGAUCTACAC (SEQ ID |
| | NO: 17) |
| PS-BA(−) | /5PS/-TTTTTTTTTTCAAGCAGAAGACGGCATACGAGAT (SEQ ID NO: 18) |
| BA(+) | AATGATACGGCGACCACCGAGAUCTACAC (SEQ ID NO: 19) |
| BA(−) | CAAGCAGAAGACGGCATACGAGAT (SEQ ID NO: 20) |
| Cy5-probe | /5Cy5/-CTTGCGCTAGTCCGGCAACCTCGGTGG (SEQ ID NO: 21) |
| UMI-primer | CTACACGACGCTCTTCCGATCTNNNNNNNCCGCTAATACGACTCACTA |
| | TAGGG (SEQ ID NO: 22) |
| TruSeq | CCTACACGACGCTCTTCCGATC (SEQ ID NO: 23) |
| sequencing primer | |
| In-house polony | TTCCATCATTGTTCCCGGGTTCCTCATTCTC (SEQ ID NO: 24) |
| sequencing primer | |
| TSO primer | /5′BIOSG/AAGCAGTGGTATCAACGCAGAGT (SEQ ID NO: 25) |
| TSO oligo | AAGCTGGTATCAACGCAGAGTGAATrG+GrG (SEQ ID NO: 26) |
| TruSeq libP5 | AATGATACGGCGACCACCGAGATCTACACTCTTTCCCTACACGACGCT |
| primer | CTTCCGATCT (SEQ ID NO: 27) |
| Nextra index | CAAGCAGAAGACGGCATACGAGAT[INDEX]GTCTCGTGGGCTCGG |
| primer | (SEQ ID NO: 28) |

“r” prior to base indicates ribonucleic base;
“+” indicates locked nucleic acid (LNA);
“5′PS” indicates 5′ phosphorothioate modification.

### Gel Casting

Polony gels were casted on 75×25 mm² glass slides (Fisher Scientific, 12-550D) or 40-mm-diameter round coverslips (#1.5; Warner Instruments, 64-1696), which were cleaned by sonication in 5% CONTRAD 70, 0.5 M NaOH, 0.1 N HCl, and Milli-Q H₂O, air dried in an AirClean PCR hood, and coated with Bind-silane (Sigma M6514) as previously described (17). To form a gel casting well on the slides, adhesive tapes (Grainer Carton Sealing Tape, 1.6 mil) with a punched center hole of 62×12 mm² were attached to each slide. The casting of the crosslinked polyacrylamide was performed in an anaerobic chamber (Coy Lab) as previously described (17). Briefly, a gel casting solution comprising 8% acrylamide/bis-acrylamide (w/v, 19:2; Sigma-Aldrich, A9099 and M7279), 16 mg/mL N-(5-bromoacetamidylpentyl) acrylamide (Combi-blocks, HD-8626), 0.015% (w/v) ammonium persulfate (freshly dissolved; Sigma-Aldrich, A3678), and 0.025% (v/v) N,N,N N-tetramethylethylenediamine (Thermo Fisher Scientific, 15524-010) was mixed and immediately pipetted into the well covered with a 60×24 mm cover glass (Corning, 2940-246). About 40 μL casting solution was typically used to cast a 40-μm-thick gel. Gels were polymerized at room temperature (R.T.) in the anaerobic chamber for 2 hours before transferred to a PCR hood. After removing the coverslip, a gel-coated slide was assembled into a modified flowcell (BioSurface Technologies, FC 81-PC) forming a channel of 55×9×0.3 mm³ with a volume of about 150 μL. Gels were casted on the round coverslips for sequencing spatial barcodes on the gels. Typically, <5-μm-thick gels were formed by placing a cut glass slide of 8×40 mm² atop a gel casting solution applied to the coverslip surface to form a uniform liquid layer between the coverslip and the top glass slide. The gels were polymerized in the anaerobic chamber at R.T. for 90 minutes. After removing the top glass, the gel-coated coverslip was assembled into a FCS2 flowcell (Bioptechs) with a 0.2-mm-thick gasket to form a channel of 8×35 mm² with a volume of about 60 L.

### Primer Grafting

A flowcell was washed with 1 mL MILLI-Q water and then 500 μL grafting buffer (10 mM potassium phosphate buffer, pH 7). Primer grafting was performed by incubating 25 μM each 5′ phosphorothioate-modified primers (PS-BA(+) and PS-BA(−); Table 1) in the grafting buffer at 50° C. for 1 hour. The flowcell was washed with 500 μL hybridization buffer (5×SSC, 0.05% TWEEN-20; Invitrogen, AM9763 and Sigma-Aldrich, P9416) to remove non-hybridized primers and stored at 4° C. or used for the next step.

### Preparation of Stamp Gels

To seed DNA templates on primer-grafted gels, 1 nM templates were denatured in freshly diluted 0.2 N sodium hydroxide (Sigma-Aldrich, 72068), neutralized with 200 mM Tris-HCl, pH 7, and diluted with ice-cold hybridization buffer to a working concentration of 8-12 pM, which typically resulted in a feature density of about 0.6 to 0.8 million per mm². Template hybridization to the gel was performed by adding 500 μL diluted templates into a grafted flowcell, which was incubated on a heating/cooling block (CPAC HT 2-TEC, Inheco) at 75° C. for 2 min, air cooled to 40° C., and washed with 500 μL amplification buffer (2 M betaine, 20 mM Tris-HCl (pH 8.8), 10 mM ammonium sulfate (Sigma-Aldrich A4418), 2 mM magnesium sulfate (Sigma-Aldrich, M2773), 0.1% (v/v) Triton-X-100 (Sigma-Aldrich T8787), and 1.3% (v/v) DMSO (Sigma-Aldrich, D8418)) to remove non-hybridized templates. To synthesize the first-strand DNAs anchored to the gel, the flowcell was added with 150 μL Taq DNA polymerase mixture (50 U/mL Taq DNA polymerase (NEB M0267) and 200 μM each dNTPs (GenScript C01582)) in the amplification buffer and incubated at 74° C. for 5 min before decreasing the temperature to 60° C. for bridge amplification. Polony amplification was performed with an automated fluidic device with a P625 pump set (Instech Laboratories). The amplification was performed at 60° C. with a HT 2-TEC heating/cooling block for 22 cycles, each including i) Denaturation: pump 500 μL deionized formamide (Emdmillipore, 4670) and stop for 1 min; ii) Annealing: pump 500 μL amplification buffer and stop for 2 seconds; iii) Extension: pump 500 μL Bst DNA polymerase mixture (80 U/mL Bst DNA polymerase (NEB, M0275 or lab purified with the same performance) and 200 μM each dNTPs) in the amplification buffer and stop for 1 minute. The flow rate was 3 mL/minute. After the amplification, double-stranded polony DNAs were linearized by washing the flowcell with 500 μL 1× CUTSMART buffer (NEB), adding 150 μL 100 U/mL USER (NEB M5505) in 1× CUTSMART buffer, and the reaction was incubated at 37° C. for 1 hour. Gels with linearized DNAs were stored in 100% formamide or washed with 500 μL elution buffer (1×SSC and 70% formamide (v/v)) before the stamping.

### Gel Stamping

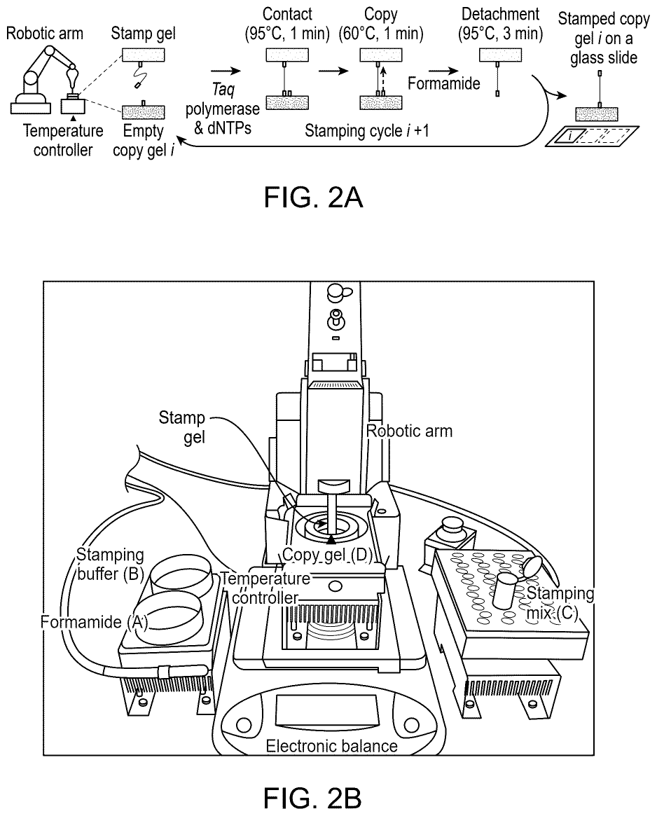

Slides with primer-grafted gels were used as copy gels. Slides with stamp gels were cut by a glass cutter to the stamps of specific sizes (e.g., 7×7 mm²). A stamp was fixed by double-sided tape (3M, 468MP) to a flat surface (φ8 mm) of a stamp holder connected to a desktop 4-axis robotic arm (DOBOT, MG400) with the positioning repeatability of 0.05 mm. A stamping cycle included seven steps involving the placement of the stamp at four positions (A-D) (FIG. 2A and FIG. 2B): 1) In Position A, soaked the stamp in formamide at 60° C. 2) Moved the stamp from Position A to Position B to wash the stamp with a stamping buffer (2 M betaine, 20 mM Tris-HCl (pH 8.8), 10 mM ammonium sulfate, 2 mM magnesium sulfate, 0.1% (v/v) Triton-X-100, and 1.3% (v/v) DMSO) at 60° C. for 10 seconds. 3) Moved the stamp from Position B to Position C to soak the stamp in a stamp mix (100 U/mL Taq DNA polymerase and 200 μM each dNTPs in the stamping buffer) at 4° C. for 30 seconds. 4) Moved the stamp from Position C to Position D to place the stamp on a specified copy gel position with a stamping pressure between 10 and 30 kPa at 95° C. for 1 minute. Before the stamping, the copy gel was preincubated in the stamp mix at 95° C. The pressure was monitored by an electronic balance (e.g., a weight of about 50 to 150 grams applied to the stamp of about 7×7 mm²; Bonvoisin, BH523). 5) In Position D, decreased the copy gel temperature to 60° C. and held the temperature for 1 minute. 6) In Position D, increased the copy gel temperature to 95° C. and meanwhile added 2 mL formamide to soak the stamp and copy gels for 3 minutes to dissociate the double-stranded DNAs. 7) Moved the stamp from Position D to Position A to prepare for the next cycle. Meanwhile, washed the copy gel with 3×1 mL MILLI-Q H₂O to prepare it for stamping on the next gel position. Six 7×7 mm² arrays were stamped on a copy gel of 55×9 mm² and then another copy gel was placed in Position D.

### Post-Stamping Gel Processing

After the stamping, copy gels were assembled to the flowcell and copied DNAs were bridge amplified for 22 cycles similarly as described in the stamp gel preparation. To expose 3′ poly(T) probe for RNA capturing, the flowcell was washed with 500 μL 1× CUTSMART buffer (NEB), added with 160 μL 2,000 U/mL TaqI (NEB, R0149) in 1× CUTSMART buffer, and incubated at 60° C. for 1.5 hours. TaqI-treated polony gels were stored in 100% formamide or washed with 500 μL elution buffer (1×SSC and 70% formamide (v/v)) for Pixel-seq assays or polony sequencing.

### Pixel-Seq Assay

### Tissue Preparation

Mice were anesthetized with Beuthansia (0.2 mL, i.p.; Merck) and decapitated. The brain was rapidly dissected, frozen on crushed dry ice, and stored at −80° C. until cryosectioning.

Transcript Capturing and cDNA Synthesis

A polony gel slide was disassembled from the flowcell, washed with MILLI-Q water and 3×200 μL wash buffer 1 (0.1×SSC and 0.4× MAXIMA H Minus RT buffer (Thermo Fisher, EP0753)), and dried in the PCR hood before use. For cryosectioning, a frozen tissue block was equilibrated at −20° C. in a Cryostat NX70 (Thermo Scientific) for 15 minutes, mounted onto a holder with O.C.T. (Fisher Healthcare, 4585), and sliced to 10-μm sections. Immediately after placing tissue sections on printed array positions on the dry gel surface, 50 μL tissue hybridization buffer (6×SSC and 2 U/μL RNAseOUT (Thermo Fisher, 10777019) was gently applied to immerse the sections and then the gel was incubated at R.T. for 15 minutes. After the hybridization, the buffer was removed by pipetting and the polony gel slide was assembled with multi-well reaction chambers (PROPLATE; Grace Bio-Labs, 246868). cDNAs were synthesized by adding 100 μL reverse transcription (RT) mixture (5 μL MAXIMA H-reverse transcriptase (Thermo Fisher, EP0753), 20 μL 5× MAXIMA RT buffer, 20 μL 20% Ficoll PM-400 (Sigma-Aldrich, F4375), 10 μL 10 mM each dNTPs, 5 μL 50 μM template switch oligo (Qiagen, 339414YC00076714), 2.5 μL RNAseOUT (40 U/μL), and 37.5 μL H₂O) into each well and incubating the chambers at 42° C. for 1 hour. A Cy5-dCTP-labeled cDNA assay was performed at the same condition except replacing the dNTPs with 500 μM each dATP/dGTP/dTTP, 12.5 μM dCTP, and 25 μM Cy5-dCTP (PerkinElmer, NEL577001EA).

### Tissue Cleanup

After the cDNA synthesis, the reaction buffer was removed, and the tissues were washed by 3×200 μL 0.1×SSC. 100 μL proteinase K digestion solution (10 μL proteinase K (Qiagen, 19131) in 90 μL PKD buffer (Qiagen, 1034963)) was added to each chamber and incubated at 55° C. for 30 min. To remove digested proteins, genomic DNAs, and others, each chamber was washed with 3×200 μL elution buffer 2 (2×SSC and 0.1% SDS) and 3×200 μL wash buffer 2 (0.1×SSC).

### Sequencing Library Construction

Recovering spatially barcoded cDNAs from the gel and introducing unique molecular identifiers (UMIs) into cDNAs were achieved by the second-strand synthesis and primer extension, respectively. For example, 70 μL second-strand mix (7 μL 10× isothermal amplification buffer (NEB, B0357), 7 μL 10 mM each dNTP mix, 3.5 μL 10 μM TSO primer, 0.5 μL 20 mg/mL BSA (NEB, B9000), 3 μL BST2.0 WARMSTART DNA Polymerase (NEB, M0538), and 49 μL H₂O) was added to each chamber and the chambers were sealed with a sealing film. After incubating the chambers at 65° C. for 15 minutes, the reagent was removed, and the gel was washed by 3×200 μL 0.1×SSC. To elute the DNAs, a denature and elution mix (35 μL 0.05 M KOH) was added to each chamber, incubated at R.T. for 10 min, and neutralized by 5 μL Tris (1 M, pH 7.0). About 35 μL sample was transferred from each chamber into a tube and added with 65 μL UMI incorporation mix (50 μL 2×Q5 Ultra II master mix (NEB, M0544), 2.5 μL 10 μM UMI primer (Table 1), and 12.5 μL H₂O). The UMI incorporation was performed by denaturing at 95° C. for 30 seconds, annealing at 65° C. for 30 seconds, and extension at 72° C. for 5 min in a PCR machine. Non-incorporated primer was removed by incubating the sample with 1 μL thermolabile exonuclease I (20 U; NEB, M0568) at 37° C. for 4 min and inactivating the exonuclease at 80° C. for 1 minute. To amplify the cDNA library, the tube was added with 2 μL each 10 μM TSO and TruSeq libP5 sequencing primers (Table 1) and 1 μL Q5 HotStart polymerase (NEB M0493) for PCR amplification to obtain 5-10 ng DNA per sample. PCR reactions were performed as follows: annealing at 95° C. for 3 min, 4 amplification cycles each including 98° C. for 20 seconds, 65° C. for 45 seconds, and 72° C. for 3 min, 8 amplification cycles each including 98° C. for 20 seconds, 67° C. for 20 seconds, and 72° C. for 3 min, and a final incubation at 72° C. for 5 minutes. Twelve PCR cycles were used to amplify cDNAs from an OB section. Typically, after the amplification, about1 ng DNA was used for sequencing library construction with a Nextera XT kit (Illumina FC-131-1024) and the TruSeq LibP5 primer, and Nextera index primers (Table 1) following the manufacturer's protocol.

### Polony Sequencing

To determine barcode sequences and distribution, the gel stamping was performed with a stamp and a copy gel on a round coverslip similarly as described above. The coverslip was assembled into a FCS2 flowcell for the polony sequencing with a HiSeq SBS kit v4 (Illumina, FC-401-4002). Images were acquired using a Nikon Ti-E automated inverted epifluorescence microscope equipped with a perfect focus system, a Nikon CFI60 Plan Fluor 40×/1.3-NA oil immersion objective, a linear encoded motorized stage (Nikon Ti-S-ER), and an Andor iXon Ultra 888 EMCCD camera (16-bit dynamic range, 1,024×1,024 array with 13-μm pixels). A four-channel imaging setup comprised two laser lines (Laser Quantum GEM 532 nm (500 mW) and Melles Griot 85-RCA-400 660 nm (400 mWS)) and two filter cubes with an emission filter (610/60-730/60 or 555/40-685/20; Chroma Technology) and a 532/660 dichroic mirror (Chroma Technology). Sequencing regents were added to the flowcell by a fluidic system comprising a multi-position microelectric valve (Valco Instruments EMH2CA) and a multi-channel syringe drive pump (Kloehn V6 12K). The sequencing was automated by building an application in Java 1.6 using jSerialComm (website: fazecast.github.io/jSerialComm/) to control the fluidic system and Micro-Manager vl.4.22 (website: micro-manager.org) to acquire images from selected stage positions. The sequencing was performed with reagents provided in the HiSeq SBS kit following a standard HiSeq sequencing protocol. Each sequencing cycle included: i) pre-cleavage wash with a cleavage buffer; ii) dye and protection group removal by a cleavage mix; iii) post-cleavage wash with a high salt buffer; iv) a pre-incorporation wash with an incorporation buffer; v) incorporation with an incorporation mix; and vi) imaging acquisition in a scan mix.

### Template Diffusion Analysis

To assess template diffusion in the RNA capturing, a mouse OB section was placed on a dried gel pre-soaked with a nucleus staining buffer (0.1×SSC, 2.5× SYTOX Green, 0.4× RT buffer) and SYTOX-stained nuclei were immediately imaged with the polony sequencing epifluorescence microscope in a FITC channel (Ex488 nm/Em520 nm). Cy5-labelled cDNAs were synthesized on the same tissue following the protocol described above and then imaged at a Cy5 channel (Ex640 nm/Em665 nm). The nucleus image served a reference for analyzing template diffusion in the Cy5 image. 10 regions (250×250 pixels, 1 pixel=0.65 μm) were randomly selected from the two images to determine a transformation matrix and a scaling factor using a MATLAB built-in function, imregister.

### Data Analysis

### Polony Image Analysis

To compare stamped polonies on different copy gels, polony images were registered to the one with the highest signal-to-noise ratio using imregcorr in MATLAB. Polonies were detected by a local-threshold method and their intensities, sizes, and centroids were measured using regionprops. A consensus map of polonies was first built with a normalized intensity >0.1 and detected in at least two out of three copy gel images. Polony center shifts were calculated as the Euclidean distance between detected polonies and the consensus.

### Base Calling of Polony Sequencing

Raw sequencing tiff images were processed to extract intensities by Dlight, a custom-built suite in MATLAB. All images were registered to the merged images of the Cy3 and Cy5 channels from the first sequencing cycle using imregcorr. Next, a polony reference map was generated from images of the first eight sequencing cycles, termed template cycles. Polonies were identified by searching local signal thresholds (>median+2× standard deviation) in all template cycle images and then finding polony centroids with an optimal chastity value. A PhiX control library (Illumina FC-110-3001) was used to optimize Dlight parameters. Intensity values of polony centroids and image pixels were analyzed by a 3Dec base-caller (36) allowing the correction of the signal crosstalk between adjacent polonies. To find polony boundaries, unassigned image pixels were compared with spatial barcode-assigned polony centroids within a distance less than 5 pixels. These pixels were assigned with adjacent assigned spatial barcodes with the highest signal correlation coefficient above 0.7. A final spatial barcode map was constructed by combining all sequenced gel positions into a single image.

### Transcript Mapping

After cDNA library sequencing, FASTQ files were processed to map transcripts to the spatial barcode map. Spatial barcode and UMI sequences were first extracted by Flexbar (37). The index sequences were mapped back to the spatial barcode map by Bowtie (38) allowing up to 2 mismatches. Paired-end reads of mapped indices were aligned to mouse transcriptome (GRCm38) using STARv2.7.0 (39) with a default setting. Sequencing reads with the same transcriptome mapping locus, UMI, and spatial barcode were collapsed to unique records for subsequent analysis.

### Results

### Polony Gels Enabled Amplifiable DNA Stamping and Show a Continuous Feature Distribution

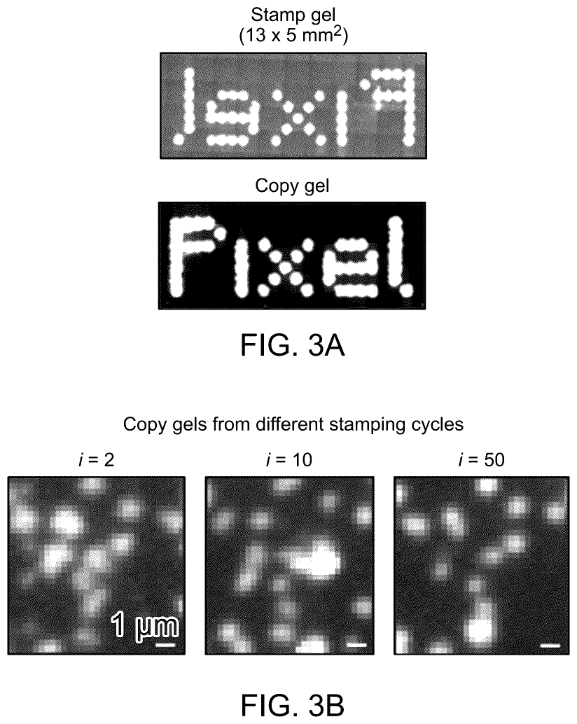

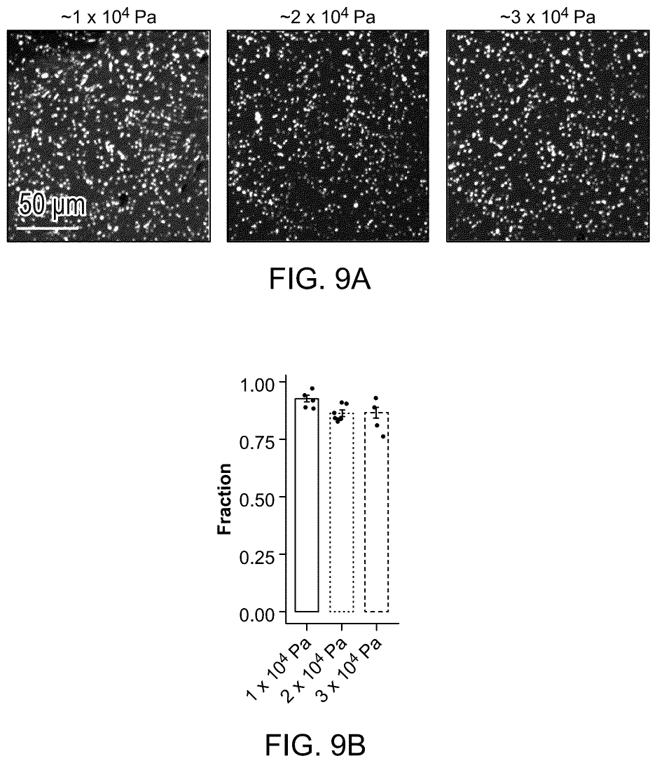

Crosslinked polyacrylamide was selected as stamp and copy gels allowing the low-pressure, conformal contact and the bridge amplification of template and copied DNAs. Different from previous methods that generated gel-embedded polonies (16, 17), polonies were amplified on gel surfaces to facilitate DNA replication between gels. To automate the stamping process, a benchtop device was built with a robotic arm to position the stamp, a thermocycler to control the gel temperature, a digital balance to monitor the stamping pressure, and a fluidic system to amplify DNAs (FIG. 2A and FIG. 2B). Gels of varied thicknesses attached to different sized glass coverslips and slides were initially compared; the efficient DNA copying between large gel areas was observed at increased gel thicknesses (e.g., 40 to 100 μm; FIG. 3A). To test the reproducibility and robustness, the stamping was consecutively performed for 50 cycles. Feature patterns found on the copy gels were consistent (FIG. 3B) and stable at varied stamping pressures (FIGS. 9A-9C).

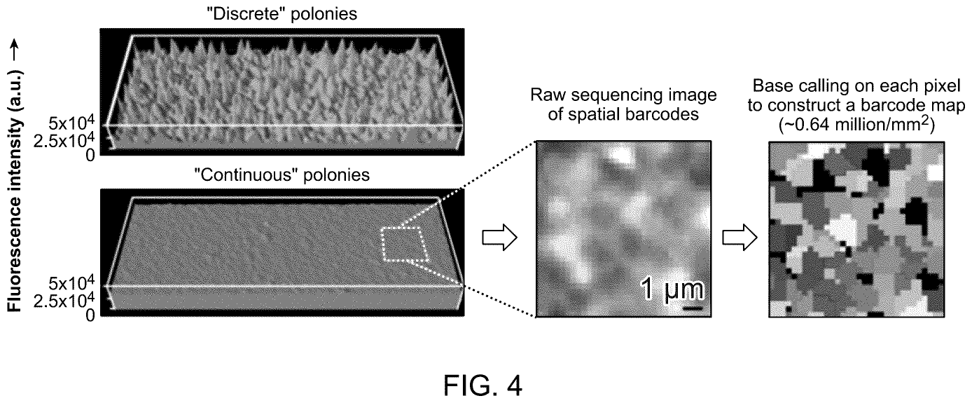

High-density polonies (>0.6 million/mm²) often form a continuous DNA distribution with minimal feature-to-feature gaps, which is distinct from those amplified in Illumina nonpatterned flowcells showing a discrete, peak-shape distribution (FIG. 4, left). To understand the difference, polonies on gel surfaces were found to have a faster size expansion likely due to decreased gel constraints on the bridge amplification. These polonies appeared to be easily accessible to restriction digestion; 93.6% of double-stranded DNAs were digested by TaqI to expose a 3′ poly(T) probe. For spatial transcriptomic assays, the continuously distributed poly(T) probes can minimize the uneven tissue RNA capture across the arrays. Although the polonies are connected, they rarely interpenetrate due to a polony exclusion effect (31); their borders can be delineated by sequencing the gel (FIG. 4 (middle)). Because polonies have varied sizes and shapes, to maximize the feature resolution, a base-calling pipeline was developed to determine the major barcode species in each pixel (0.325×0.325 μm²) of gel images to construct a spatial barcode map (FIG. 4 (right).

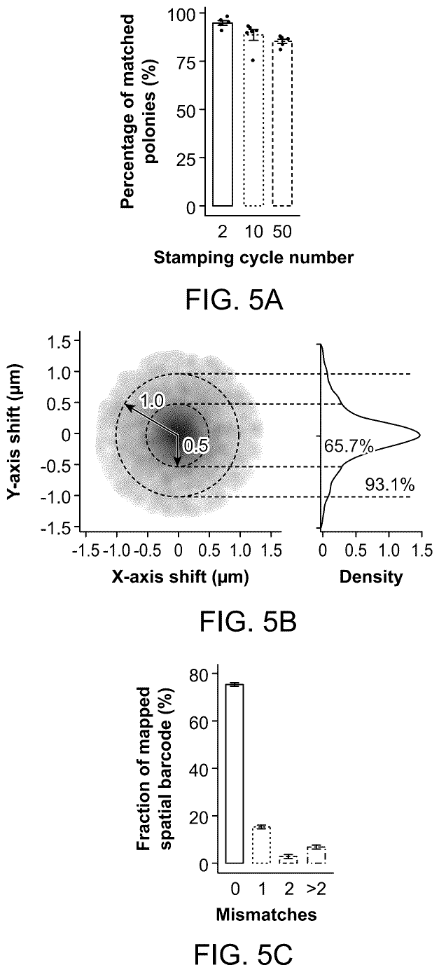

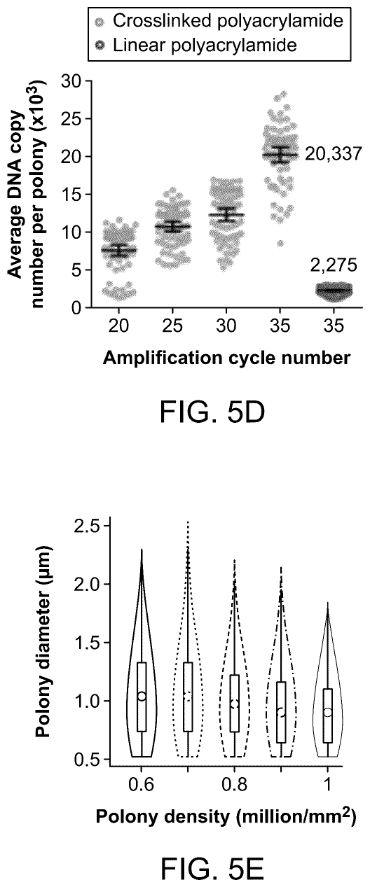

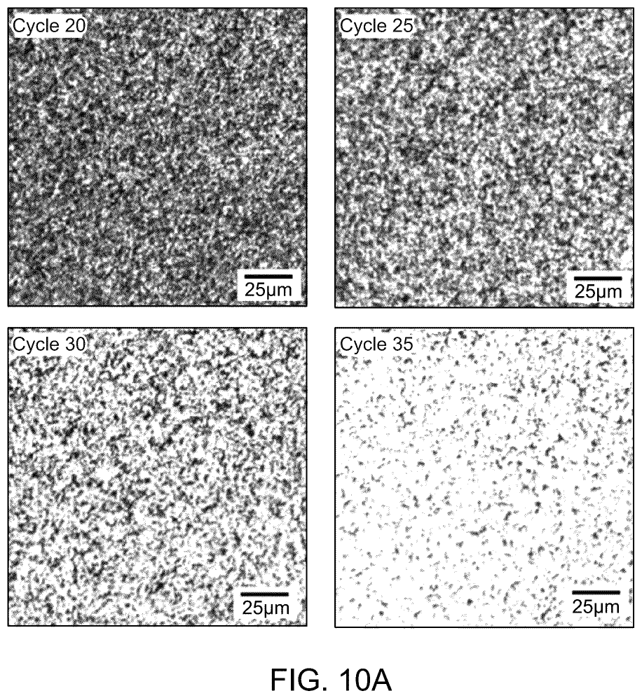

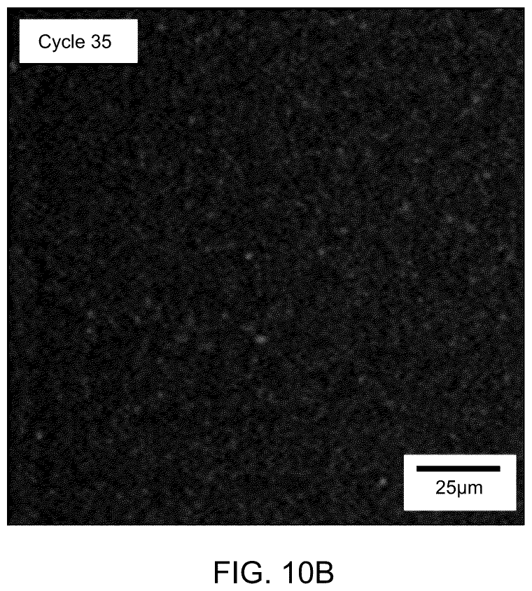

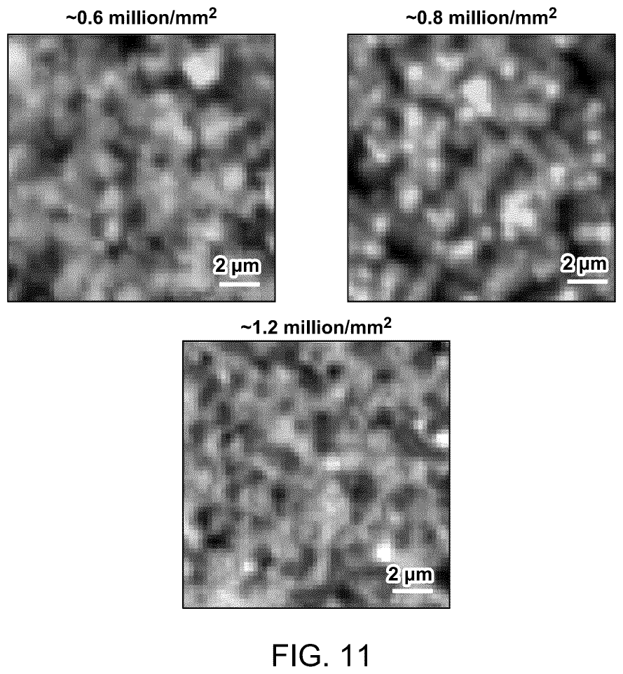

The efficient replication of polony gels requires the post-stamping bridge amplification of copied DNAs which increases the DNA densities and compensates for the inefficient copying in some gel areas. However, more amplification can cause polony size expansion and introduce errors to spatial barcodes, compromising the resolution and accuracy, respectively. To assess this issue, copy gels fabricated in a consecutive stamping experiment were quantitively compared by analyzing feature patterns in multiple gel regions. Individual gels were compared with a consensus feature map constructed with aligned images of three copy gels. The repeated stamping is robust and lost <15% of features after 50 cycles, likely due to the slow loss of templates on the stamp (FIG. 5A). Matched polonies in the gels were extracted to measure their center drifts from the consensus, which exhibit a normal distribution with 93.1% and 65.7% of the center distances below 1 and 0.5 μm, respectively (FIG. 5B). By sequencing 24-base pair spatial barcodes, 93.43±0.04% of matched polonies in two gels were found with matched spatial barcodes with up to two mismatched bases (FIG. 5C). Amplified polonies contained ultradense capture probes; for example, the amplification yielded an average of 20,337 template copies per polony after 35 cycles (FIG. 5D and FIGS. 10A-10B), an about 9-fold increase from an Illumina method (18). With the sequencing imaging setup, gels were reliably fabricated with about 0.6 to 0.8 million features per mm² passing filter and a mean feature diameter of 1.07 to 0.906 μm (FIG. 5E). Fabricating higher resolution gels with smaller and denser features is possible because even more crowded polonies still show clear boundaries (FIG. 11) but sequencing them requires improved imaging resolution.

### Polony Gel-Enabled Spatial Transcriptomics Shows the High Resolution and Sensitivity

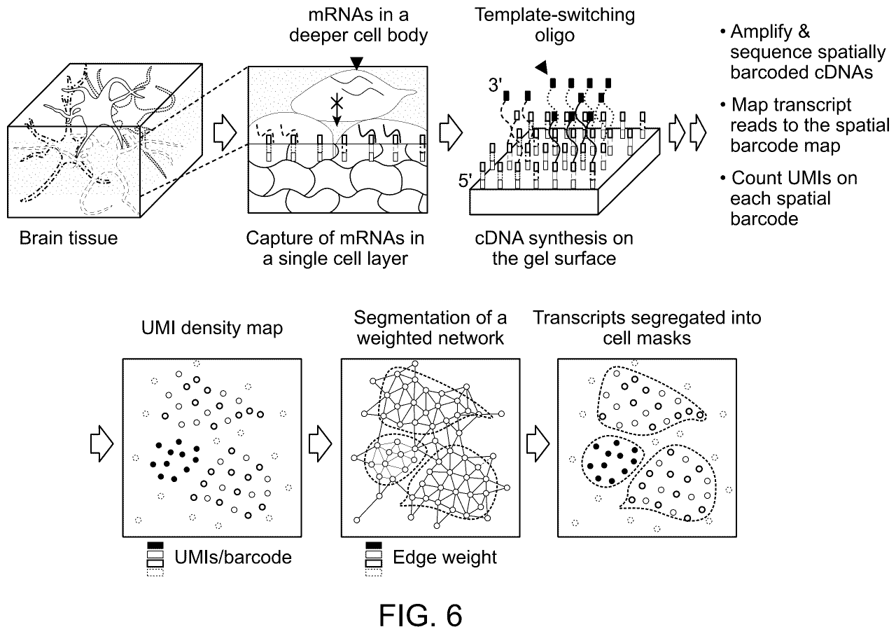

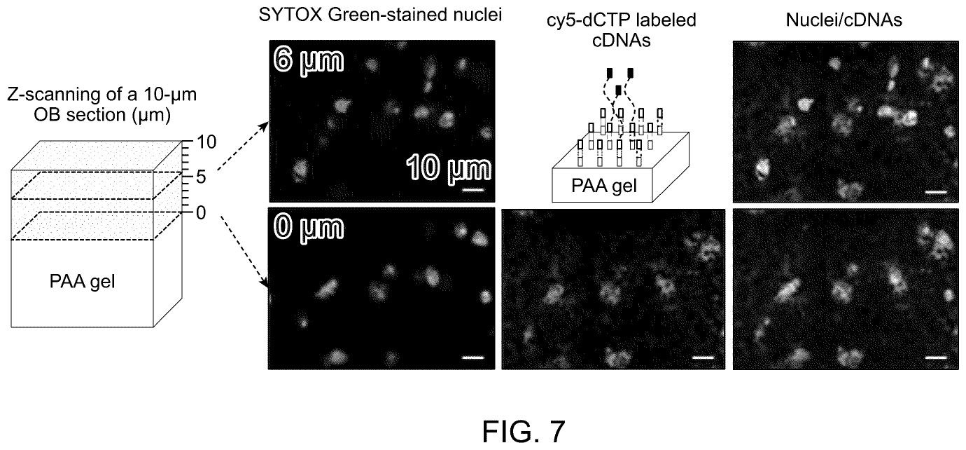

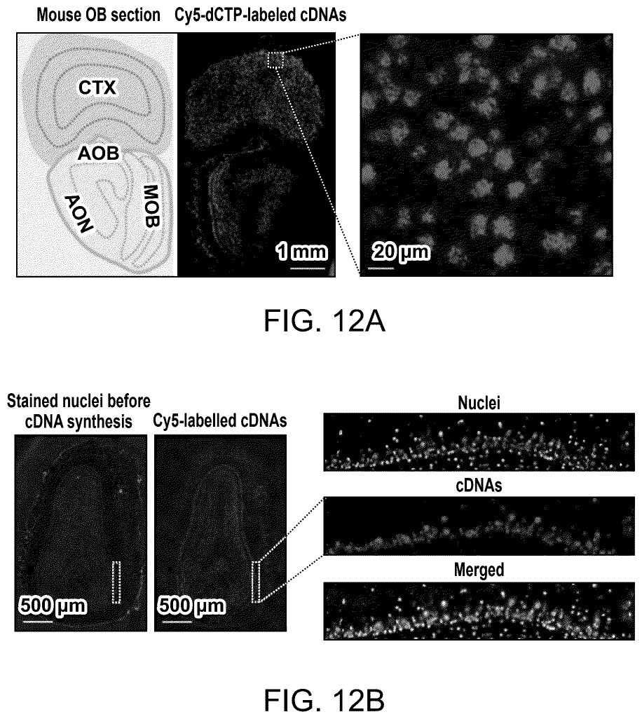

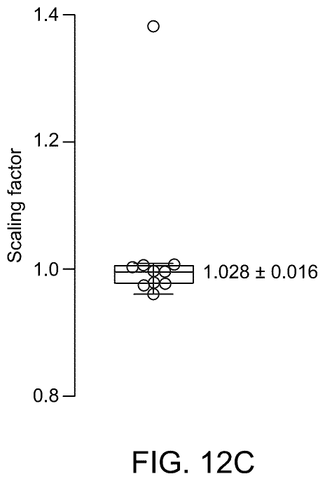

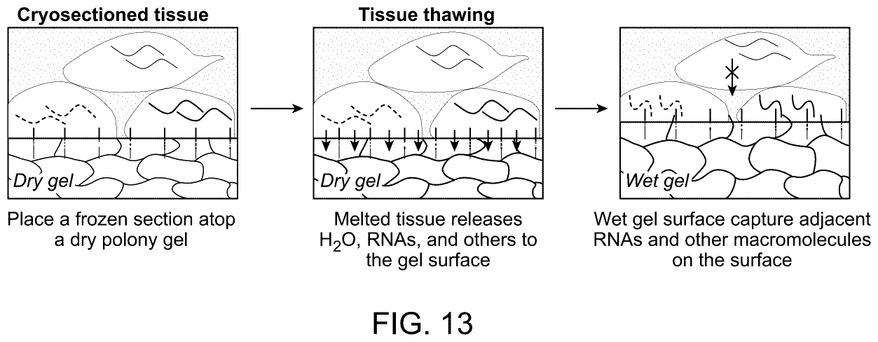

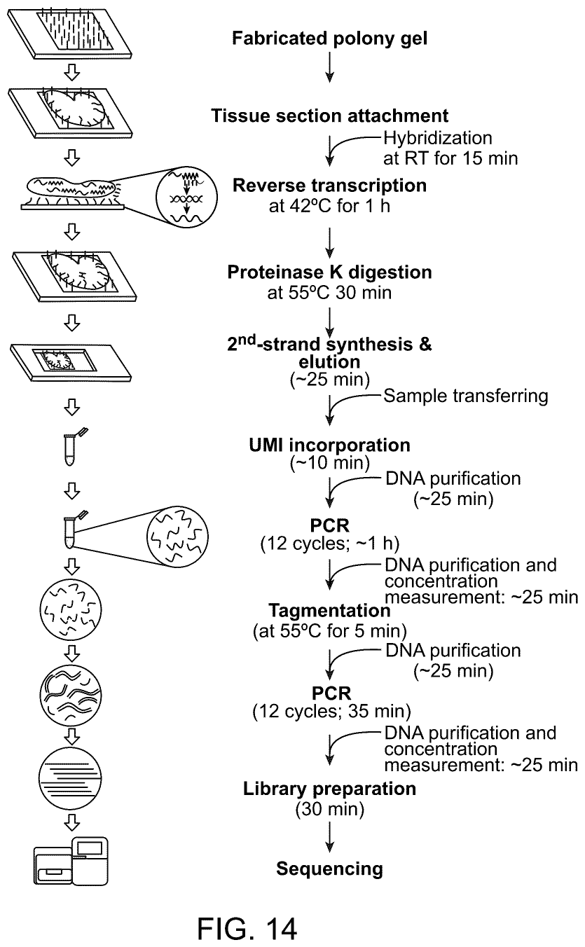

Pixel-seq was developed with a focus on translating the 1-μm feature resolution of the gels to the single-cell resolution of the assay for mapping complex tissues such as the brain (FIG. 6). To test assay conditions and compare the performance, the mouse olfactory bulb (OB) was analyzed with morphologically diverse cells organized in a layered structure often used to validate spatial transcriptomic assays (13, 14, 23, 24, 26). Two common issues of the array-based assays limiting the single-cell resolution were investigated: the lateral diffusion of RNAs between cells and the mixing of RNAs from multilayered cells found even in thin tissue sections. The polony gel-based RNA capturing, even without tissue fixation, yielded strong complementary DNAs (cDNAs) signals clearly delineating boundaries of neuronal cell bodies with minimal template drift (FIGS. 12A-12C), indicating that the gel substrate can largely constrain the lateral diffusion of RNAs on its surface. Additionally, the gels appeared to capture tissue RNAs from a single cell layer when frozen sections were placed on the dried gels; yielded Cy5-labelled cDNA signals arising from the captured mRNAs were colocalized with nuclei in the gel-contacting layer of cells not those in a deep layer (FIG. 7). The selective RNA capturing can be explained by fast occupancy of a gel surface by adjacent RNAs during the gel wetting by a tissue section (FIG. 13). The gel-based capturing not only increased the resolution but also facilitated the fast preparation of cDNA sequencing libraries (about 6 hours; FIG. 14).

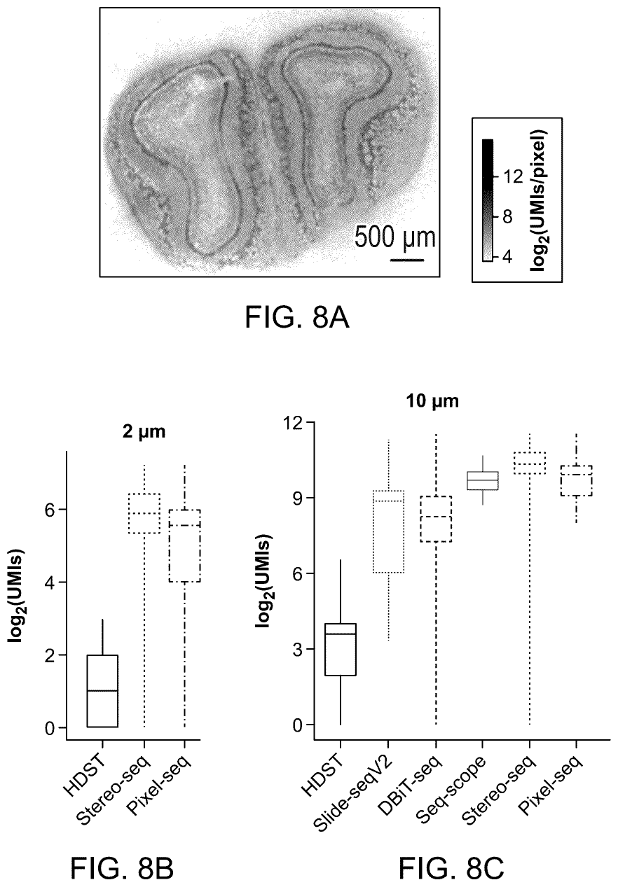

To assess the performance, 10-μm, coronal OB sections were assayed to obtain spatially resolved transcriptomes. For example, in an about 13-mm² OB section, about 83% of raw reads were mapped to the barcode map to obtain about 82.5 million UMIs with a density range from 1 to 678 UMIs/barcode. The UMI density map shows a continuous, pixel-resolution, multi-layered organization of cell bodies (FIG. 8A). About 23,000 unique genes were detected with over 10 UMIs in at least one of three replicates; the data showed high correlation (R≥0.968). Although the assay only captured RNAs in a gel-contacting cell layer instead of a whole 10-μm section, Pixel-seq gave a similar capture efficiency as the top-performing methods (FIG. 8B and Table 2); for example, with a sequencing depth of about 80%, Pixel-seq detected, in a whole OB, a mean of 47 and 977 UMIs per bin of 2×2 and 10×10 μm², respectively, and in regions with densely populated cell bodies such as a mitral cell layer (MCL), a mean of 83 and 1,695 UMIs per bin of 2×2 and 10×10 μm², respectively.

Copying DNA Cluster Arrays from an Illumina NovaSeq Flowcell to a Polony Gel

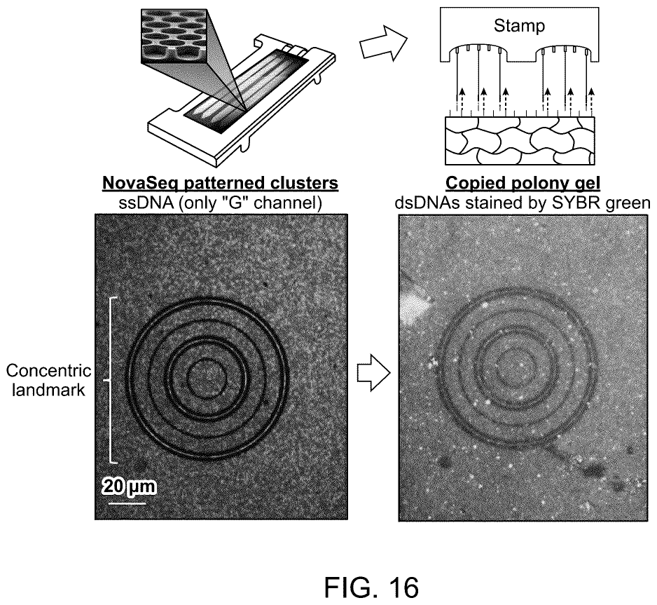

Polony gel stamping not only can copy DNA arrays between two gel surfaces, but also can copy DNA arrays between a rigid surface and an elastic gel. The rigid surface can even have chemically or photo etched microwell or nanowell structures because the elasticity of a gel can conformationally contact inner surfaces of microwells or nanowells to copy DNAs. It was demonstrated that DNA templates in an IlluminaNovaSeq flowcell were efficiently copied to a polony gel with matched concentric landmarks (FIG. 16). Copying patterned DNA clusters to polony gels not only increased polony gel feature resolution from 1 to 0.65 μm or even less, but also offered a significant high RNA capture efficiency than that achieved with nanowell sequencing flowcells. DNA probes in nanowells have much lower molecular density and decreased accessibility to tissue RNAs.

TABLE 2
Comparison of array-based Spatial Transcriptomics (ST) methods

| | | | | | Percentage | |
|---|---|---|---|---|---|---|
| | | Barcode | | Feature | of array | Feature |
| | | sequencing | Feature | center | area | density |
| ST | DNA | on each | diameter(s) | distance(s) | covered by | (features |
| method | features | array | (μm) | (μm) | features^{a} | per mm²) |
| Visium | Spotted | No | 55 | 100 | 27% | ~110 |
| | DNAs | | | | | |
| DBiT-Seq | Microfluidic | No | 1) 10 | 1) 30 | 1) 11% | 1) 1,110 |
| | wells | | 2) 25 | 2) 53 | 2) 22% | 2) 360 |
| | | | 3) 50 | 3) 100 | 3) 25% | 3) 100 |
| Slide- | Assembled | Yes | 10 | 10 | 78% | 1 × 10⁴ |
| seqV2 | beads | | | | | |
| HDST | Assembled | Yes | 2 | 3 | 34% | 1.07 × 10⁵ |
| | beads | | | | | |
| Seq-scope | Illumina | Yes | ≥0.5 | ≤0.8 | <70% | ≤1.5 × 10⁶ |
| | DNA | | | | | |
| | clusters | | | | | |
| Stereo-seq | DNA | Yes | 0.22 | 1) 0.5 | 1) 15% | 1) 4 × 10⁶ |
| | nanoballs | | | 2) 0.715 | 2) 7% | 2) 1.96 × 10⁶ |
| Pixel-seq | Polonies | No | ~1 | ~1 | >90% | ≤1 × 10⁶ |

| | | Tissue used for | CDNA | | | |
|---|---|---|---|---|---|---|
| ST | Array | RNA | synthesis & | | | |
| method | substrate^{b} | capturing^{c} | amplification^{d} | Mean UMIs^{e} | | Data Set |
| Visium | Glass | Mouse OB, 10 | TSO & PCR | 15,377/55 | μm | GSE153859 |
| | | μm-thickness, | | | | (10.1101/ |
| | | tissue fixation & | | | | 2020.08.24.252296) |
| | | permealization | | | | |
| DBiT-Seq | PDMS | Mouse embryo, | TSO & PCR | ~5,000/10 | μm | GSE137986 |
| | | 7 μm-thickness, | | | | (10.1016/ |
| | | tissue fixation & | | | | j.cell.2020.10.026) |
| | | permealization | | | | |
| Slide-seqV2 | Glass | Mouse OB, 10 | TSO & MDA | 497/10 | μm | (10.1038/ |
| | | μm-thickness, | | | | s41587-020-0739-1) |
| | | fresh frozen | | | | |
| HDST | Silicon | Mouse OB, 10 | T7 aRNA | 12/10 μm or | | GSE130682 |
| | | μm-thickness, | | 2/2 μm | | (10.1038/ |
| | | tissue fixation & | | | | s41592-019-0548-y |
| | | permealization | | | | |
| Seq-scope | Linear PAA | Mouse colon, | TSO & MDA | ~1000/10 | μm | GSE169706 |
| | coating | 10 μm-thickness, | | | | (10.1016/ |
| | | tissue fixation & | | | | j.cell.2021.05.010) |
| | | permealization | | | | |

| | | | | | |
|---|---|---|---|---|---|
| Stereo-seq | Silicon | Mouse OB, 10 | TSO & MDA | ~1450/10 μm | GSE153164 |
| | | μm-thickness, | | or 59/2 μm | (10.1016/ |
| | | tissue fixation & | | | j.cell.2022.04.003) |
| | | permealization | | | |
| Pixel-seq | Crosslinked | Mouse OB, 10 | TSO & PCR | 977/10 μm or | GSE186097 |
| | PAA gel | μm-thickness, | | 47/2 μm | |
| | | fresh frozen | | | |

^{a}The percentages for Visium, DBiT-Seq, Slide-seqV2, HDST, and Stereo-seq were calculated based on reported feature sizes, densities, and spatial patterns. The percentage for Seq-scope was measured by analyzing a reported cluster image of the flowcell hybridized with the highest template concentration, 100 mM.
^{b}PAA, polyacrylamide
^{c}Different RNA capturing conditions were used and some likely captured RNAs from multiple cell layers from a whole tissue section.
^{d}TSO, template-switching oligo; MDA, multiple-displacement amplification; T7 aRNA, T7 RNA polymerase-based amplification. Different amplification methods have different yields. MDA amplifies cDNA fragments and typically gives a higher yield than the PCR amplification of full-length cDNAs.
^{e}UMI counts were obtained from the original publications. Feature gaps typically were not considered in the calculations.

### DISCUSSION

This disclosure demonstrates the scalable, fast, low-cost fabrication of the 1-μm-resolution clonal DNA cluster arrays. For example, the consumable cost of fabricating a 7×7 mm² array of >30 million unique features decreased to about $3 (Table 3) and the time to about 6 hours.

TABLE 3
Consumable list for polony gel fabrication

| | | | | | | | | | Cost ($)/gel |
|---|---|---|---|---|---|---|---|---|---|
| | | | | | | | | | (e.g., gel |
| | | | | | | | | | area of ~55*9 |
| | | | | | | UW | | | mm² printed |
| Fabrication | Reagents & | | | | | price | | | with six 7*7 |
| step | consumables | Vendor | Cat. # | Size | | ($) | Amount | | mm² arrays) |
| Glass | Microscope | Fisher | 12-550D | 144 | pieces/pack | 19.95 | 1 piece/gel | | 0.14 |
| cleaning | slide | Scientific | | | | | | | |
| | Contrad 70 | Decon | 1003 | 1.33 | Gallon | 141.41 | 25 mL/60 | gels | 0.01 |
| Glass | 200 Proof | Decon | 2701 | 4 × 4 | Gallon | 58.53 | 360 mL/60 | gels | 0.01 |
| surface | Ethanol | | | | | | | | |
| modification | Bind-Siline | Sigma | M6514 | 50 | mL | 88.90 | 2 mL/60 | gels | 0.06 |
| | Acetic Acid | Fisher | A35-500 | 500 | mL | 33.92 | 4 mL/60 | gels | 0.0 |
| | | Scientific | | | | | | | |
| Gel casting | Acrylamide | Sigma- | A9099 | 100 | g | 76.07 | 2 g/200 | gels | 0.01 |
| | | Aldrich | | | | | | | |
| | N,N′-methylene- | Sigma- | M7279 | 100 | g | 145.00 | 0.5 g/200 | gels | 0.0 |
| | bisacryl | Aldrich | | | | | | | |
| | amide | | | | | | | | |
| | Ammonium | Sigma- | A3678 | 25 | g | 32.20 | 100 mg/10 | gels | 0.01 |
| | persulfate | Aldrich | | | | | | | |
| | TEMED | Invitrogen | 15524-010 | 30 | mL | 48.09 | 10 μL/10 | gels | 0.01 |
| | N-(5-bromo- | Combi- | HD-8626 | 1 | g | 3,600.00 | 70 mg/100 | gels | 2.52 |
| | acetamidyl | blocks | | | | | | | |
| | pentyl) | | | | | | | | |
| | acrylamide | | | | | | | | |
| | N, N- | Sigma- | D4551 | 250 | mL | 42.70 | 500 μL/200 | gels | 0.01 |
| | dimethyl- | Aldrich | | | | | | | |
| | formamide | | | | | | | | |
| Primer | Potassium | Sigma- | P9666 | 100 | g | 41.31 | 1 g/200 | gels | 0.01 |
| grafting | phosphate | Aldrich | | | | | | | |
| | dibasic | | | | | | | | |
| | trihydrate | | | | | | | | |
| | Potassium | Sigma- | P9791 | 100 | g | 26.91 | 1/200 | gels | 0.01 |
| | phosphate | Aldrich | | | | | | | |
| | monobasic | | | | | | | | |
| | Bridge | IDT | PS-BA(+) | 3 μMole | | 527.50 | 50 μL/20 | gels | 0.88 |
| | amplification | | | (=3 mL × 1 mM) | | | | | |
| | primers | | PS-BA(−) | 2.83 μMole | | 596.50 | 50 μL/20 | gels | 1.05 |
| | | | | (=2.83 mL × 1 mM) | | | | | |
| Amplification | Betaine | Sigma- | B2629 | 1 | Kg | 176.00 | 234.3 g/20 | gels | 2.06 |
| Buffer | | Aldrich | | | | | | | |
| making | Trizma base | Sigma- | 93362 | 500 | g | 266.00 | 2.42 g/20 | gels | 0.06 |
| | | Aldrich | | | | | | | |
| | Ammonium | Sigma- | A4418 | 500 | g | 79.51 | 1.32 g/25 | gels | 0.01 |
| | sulfate | Aldrich | | | | | | | |
| | Magnesium | Sigma- | M2773 | 500 | g | 65.60 | 0.49 g/20 | gels | 0.01 |
| | sulfate | Aldrich | | | | | | | |
| | Triton X-100 | Sigma- | T8787 | 250 | mL | 90.06 | 1 mL/20 | gels | 0.02 |
| | | Aldrich | | | | | | | |
| | Dimethyl | Sigma- | D8418 | 1 | L | 315.00 | 13 mL/20 | gels | 0.20 |
| | sulfoxide | Aldrich | | | | | | | |
| Array | dNTP (10 mM | GenScript | C01582- | 250 | mL | 2,500.00 | 30 L/20 | gels | 0.02 |
| printing | each) | | 250 | | | | | | |
| | Taq DNA | New England | M0267X | 4000 units | | 369.60 | 15 L/20 | gels | 0.35 |
| | polymerase | Biolabs | | (800 μL) | | | | | |
| Polony | Formamide | Emdmillipore | 4670-4L | 4 | L | 447.13 | 25 mL/gel | | 2.79 |
| Amplification | (Deionized) | | | | | | | | |
| | Bst Enzyme | Lab | — | — | | — | — | | <1 |
| | | purified | | | | | | | |
| | dNTP (10 mM | GenScript | C01582- | 250 | mL | 2,500.00 | 320 μL/gel | | 3.20 |
| | each) | | 250 | | | | | | |
| TaqI | Taq I-v2 | New England | R0149L | 20,000 units | | 209.60 | 16 μL/gel | | 3.35 |
| digestion | | Biolabs | | (1 mL) | | | | | |
| Total cost of the stamping method (6 arrays) | | | | $17.80 | | | | | |
| Barcode | USER enzyme | New England | M5505L | 250 units | | 248.8 | 16 μL/gel | | 15.92 |
| sequencing | | Biolabs | | (0.25 mL) | | | | | |
| (e.g., gel | Sequencing | IDT | | 50 nMole | | 136.85 | 30 μL* 100 μm/gel | | 0.82 |
| area of ~22*9 | primer | | | (=5 mL*100 μM) | | | | | |
| mm² | Illimina Hiseq | Illumina | FC-401- | 50 | cycles | 2,805.00 | 1 kit/100 | gels | 280.50 |
| printed with | V4 hit | | 4002 | | | | | | |
| three 7*7 | (Sequencing | | | | | | | | |
| mm² arrays) | reagents not | | | | | | | | |
| | including | | | | | | | | |
| | flowcell) | | | | | | | | |

| | |
|---|---|
| Total cost of the fabrication requiring | $315 |
| array sequencing (3 arrays) | |
| Cost reduction fold: | 35.39 |

The amplifiable stamping method is applicable to copy other arrays. Some spotted, in-situ synthesized, or self-assembled arrays (e.g., DNA-coated beads, DNA nanoballs, and DNA clusters in non-patterned and patterned Illumina sequencing flowcells) can serve as a stamp to copy templates from these arrays to a hydrogel surface and then the copied DNAs can be amplified by the bridge amplification to increase the DNA density. The gels with copied DNAs can be used as a stamp to make more copies.

A sequential stamping method can increase the array feature coverage and feature density. The stamping can be sequentially performed with multiple different stamps on the same copy gel position to achieve a higher array coverage and feature density, the latter of which is impossible to be analyzed by fluorescence imaging-based next-generation sequencing due to the optical resolution. Indeed, all imaging-based sequencing, such as the Illumina next-generation sequencing platforms, have the limits on the maximum feature densities (e.g., 1 million clusters/mm²). The sequential stamping can surpass the limit.

In addition to the compatibility with electroblotting, the hydrogel substrate is superior to other solid surface substrates (e.g., glass, silicone, and PDMS) not only because of its ability to restrain template diffusion and enhance the capture efficiency, but also due to its compatibility with electrophoresis.

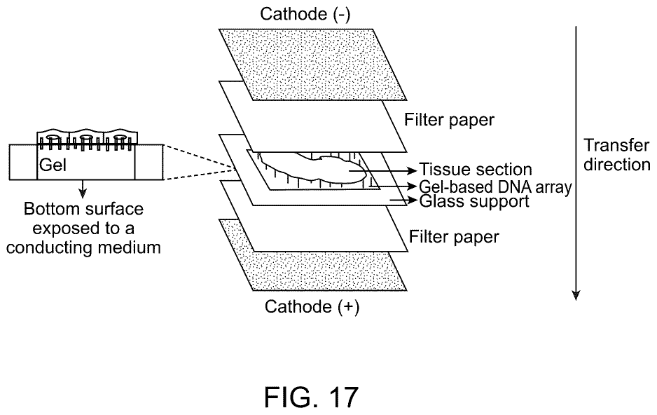

The hydrogel substrates can be coupled to electrophoresis-assisted capturing (or electroblotting like western blotting) to enhance the spatial resolution and capture efficiency (FIG. 17). DNA array-based in situ capturing of analytes in tissues typically relies on non-directional migration of tissue analytes to array probes to achieve the capturing. The non-directional migration causes lateral diffusion which prevents an assay from achieving a high spatial resolution (e.g., single-cell or subcellular resolution) and increasing loss of analytes in deeper tissues. Here, the DNA stamping method can generate replicated copy arrays on a polyacrylamide gel with the top and bottom surfaces exposed to a conducting medium, allowing ions to migrate in or out. Thus, an electric field vertical to the gel top surface can be applied to drive migration of analytes in the sample to the top surface of the gel. The gel has small pore sizes to prevent the analytes from further migrating into the gel. Target analytes (e.g., nucleic acid targets) on the gel surface are captured by hybridization to probe sequences in the array.

DNA arrays fabricated by polony gel stamping are more suitable for region-selective analysis of captured DNAs on the arrays. A major cost component of DNA array-based spatial barcoding coupled to next-generation sequencing of barcoded cDNAs is the DNA sequencing. The sequencing cost scales linearly with the tissue size and RNA detection sensitivity. However, many users are only interested in specific tissue regions and cell types. It is highly desirable to only sequence barcoded cDNAs from selected tissue regions.

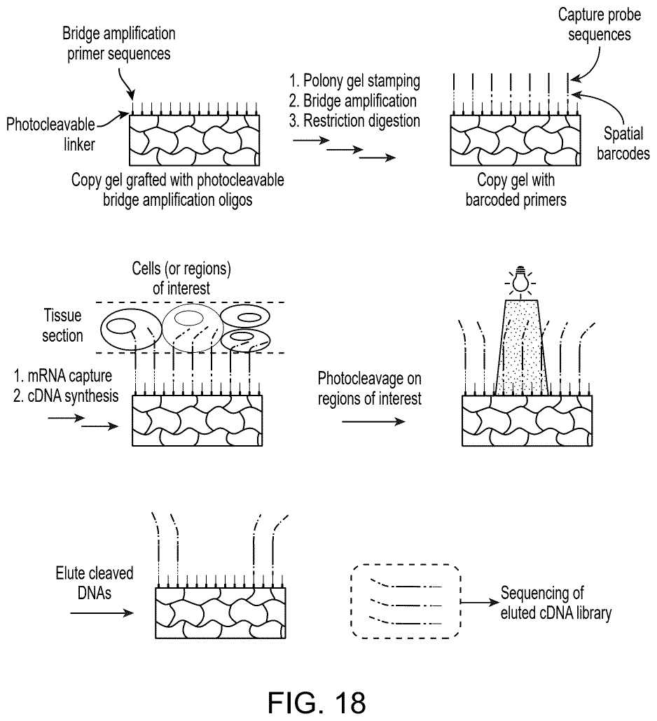

One method is to photo release barcoded cDNAs on selected gel regions by cutting a photocleavable spacer synthesized into bridge amplification primers (FIG. 18). This method can lose DNAs if DNA clusters in the gel need to be sequenced by many rounds of fluorescence imaging. However, the gel stamping method avoids the light exposure to protect photolabile primers in gels.

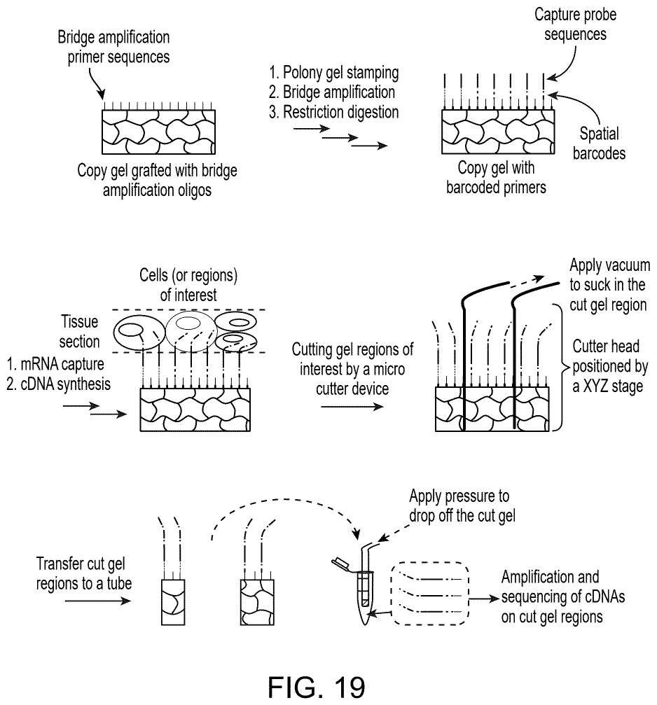

Another method is to mechanically dissect gel regions of interest because the gel is a soft material ideally suited for mechanical cutting (FIG. 19). A micro cutter device can be designed with a cutting head to dissect small gel regions, a tubing for applying vacuum and pressure to transfer cut gel pieces to a tube, and a computer controlled XYZ stage for sampling many gel regions.

In addition to polyacrylamide gels, other elastomeric materials such as other hydrogels and PDMS after surface modifications allowing the bridge amplification, can be used as the substrates for the stamping.

Polony gels can be used for spatial omics assays to detect RNA, proteins, genomic DNAs, and small molecule analytes. Polony gels were only used for spatial transcriptomics in this work, but they should be applicable to detecting proteins and epigenetic modifications with DNA-tagged antibodies (32, 33) or other affinity reagents (e.g., nanobodies, monobodies, and computationally designed binder proteins), enzyme (e.g., Tn5)-fragmented genomic DNAs, and possibly small-molecule analytes via affinity reagent innovation (34).

Beyond spatial omics application, polony gels can be designed with various probes for standard DNA microarray-based applications (35) including but not limited to gene expression and transcription factor binding, genotyping, and DNA synthesis.

### REFERENCES CITED

1. M. Schena, D. Shalon, R. W. Davis, P. O. Brown, Quantitative monitoring of gene expression patterns with a complementary DNA microarray. Science 270, 467-470 (1995).
2. J. DeRisi et al., Use of a cDNA microarray to analyse gene expression patterns in human cancer. Nat. Genet. 14, 457-460 (1996).
3. I. Barbulovic-Nad et al., Bio-microarray fabrication techniques—a review. Crit Rev Biotechnol 26, 237-259 (2006).
4. J. Khan et al., Expression profiling in cancer using cDNA microarrays. Electrophoresis 20, 223-229 (1999).
5. M. K. Deyholos, D. W. Galbraith, High-density microarrays for gene expression analysis. Cytometry 43, 229-238 (2001).
6. S. P. Fodor et al., Light-directed, spatially addressable parallel chemical synthesis. Science 251, 767-773 (1991).
7. A. C. Pease et al., Light-generated oligonucleotide arrays for rapid DNA sequence analysis. Proc Natl Acad Sci USA 91, 5022-5026 (1994).
8. M. Chee et al., Accessing genetic information with high-density DNA arrays. Science 274, 610-614 (1996).
9. R. Ammar, A. M. Smith, L. E. Heisler, G. Giaever, C. Nislow, A comparative analysis of DNA barcode microarray feature size. BMC Genomics 10, 471 (2009).
10. T. R. Hughes et al., Expression profiling using microarrays fabricated by an ink-jet oligonucleotide synthesizer. Nat Biotechnol 19, 342-347 (2001).
11. K. L. Michael, L. C. Taylor, S. L. Schultz, D. R. Walt, Randomly ordered addressable high-density optical sensor arrays. Anal Chem 70, 1242-1248 (1998).
12. K. L. Gunderson et al., Decoding randomly ordered DNA arrays. Genome Res 14, 870-877 (2004).
13. S. G. Rodriques et al., Slide-seq: A scalable technology for measuring genome-wide expression at high spatial resolution. Science 363, 1463-1467 (2019).
14. S. Vickovic et al., High-definition spatial transcriptomics for in situ tissue profiling. Nature Methods 16, 987-990 (2019).
15. R. Drmanac et al., Human Genome Sequencing Using Unchained Base Reads on Self-Assembling DNA Nanoarrays. Science 327, 78-81 (2010).
16. R. D. Mitra, G. M. Church, In situ localized amplification and contact replication of many individual DNA molecules. Nucleic Acids Res. 27, e34 (1999).
17. L. Gu et al., Multiplex single-molecule interaction profiling of DNA-barcoded proteins. Nature 515, 554-557 (2014).
18. D. R. Bentley et al., Accurate whole human genome sequencing using reversible terminator chemistry. Nature 456, 53-59 (2008).
19. J. A. Ferguson, F. J. Steemers, D. R. Walt, High-density fiber-optic DNA random microsphere array. Anal Chem 72, 5618-5624 (2000).
20. F. J. Steemers, J. A. Ferguson, D. R. Walt, Screening unlabeled DNA targets with randomly ordered fiber-optic gene arrays. Nat Biotechnol 18, 91-94 (2000).
21. D. R. Walt, Techview: molecular biology. Bead-based fiber-optic arrays. Science 287, 451-452 (2000).
22. T. Murakami, J. Sumaoka, M. Komiyama, Sensitive isothermal detection of nucleic-acid sequence by primer generation-rolling circle amplification. Nucleic Acids Res 37, e19 (2009).
23. P. L. Stahl et al., Visualization and analysis of gene expression in tissue sections by spatial transcriptomics. Science 353, 78-82 (2016).
24. R. R. Stickels et al., Highly sensitive spatial transcriptomics at near-cellular resolution with Slide-seqV2. Nat. Biotechnol. 39, 313-319 (2021).
25. C. S. Cho et al., Microscopic examination of spatial transcriptome using Seq-Scope. Cell 184, 3559-+(2021).
26. A. Chen et al., Spatiotemporal transcriptomic atlas of mouse organogenesis using DNA nanoball-patterned arrays. Cell 185, 1777-1792 (2022).
27. S. Vickovic et al., SM-Omics is an automated platform for high-throughput spatial multi-omics. Nat. Commun. 13, 795 (2022).
28. X. Fu et al., Continuous polony gels for tissue mapping with high resolution and RNA capture efficiency. bioRxiv, https://doi.org/10.1101/2021.1103.1117.435795 (2021).
29. Y. Xia, G. M. Whitesides, Soft lithography. Angew. Chem. Int. Ed. Engl. 37, 550-575 (1998).
30. S. A. Lange, V. Benes, D. P. Kern, J. K. Horber, A. Bernard, Microcontact printing of DNA molecules. Anal. Chem. 76, 1641-1647 (2004).
31. J. Aach, G. M. Church, Mathematical models of diffusion-constrained polymerase chain reactions: basis of high-throughput nucleic acid assays and simple self-organizing systems. J. Theor. Biol. 228, 31-46 (2004).
32. S. Vickovic et al., SM-Omics: An automated platform for high-throughput spatial multi-omics. bioRxiv, (2021).
33. Y. Liu et al., High-spatial-resolution multi-omics sequencing via deterministic barcoding in tissue. Cell 183, 1665-1681 (2020).
34. S. Kang et al., COMBINES-CID: An efficient method for de novo engineering of highly specific chemically induced protein dimerization systems. J. Am. Chem. Soc. 141, 10948-10952 (2019).
35. R. Bumgarner, in Curr. Protoc. Mol. Biol. (2013), chap. 22, pp. 22.21.21-22.21.11.
36. B. Wang, L. Wan, A. Wang, L. M. Li, An adaptive decorrelation method removes Illumina DNA base-calling errors caused by crosstalk between adjacent clusters. Sci. Rep. 7, (2017).
37. J. T. Roehr, C. Dieterich, K. Reinert, Flexbar 3.0-SIMD and multicore parallelization. Bioinformatics 33, 2941-2942 (2017).
38. B. Langmead, C. Trapnell, M. Pop, S. L. Salzberg, Ultrafast and memory-efficient alignment of short DNA sequences to the human genome. Genome Biology 10, (2009).
39. A. Dobin et al., STAR: ultrafast universal RNA-seq aligner. Bioinformatics 29, 15-21 (2013).

What is claimed is:

1. A method of replicating a template microarray comprising:
providing a template microarray comprising a plurality of nucleic acid features where each comprises a plurality of identical single-stranded DNA sequences that form a random or patterned array distribution;
contacting the template microarray with a copy microarray in the presence of polymerase and nucleotides to synthesize complementary DNA sequences on the copy microarray;
removing the template microarray;
amplifying synthesized complementary DNA sequences on the copy microarray to form a plurality of nucleic acid features with the sequences and distribution substantially identical to those of the template microarray; and
repeating the replicating process with the same template microarray and a plurality of copy microarrays to produce a plurality of replicated copy microarrays.

2. The method of claim 1, wherein the template microarray comprises a solid support having a top surface and a plurality of nucleic acid features on the top surface of the support.

3. The method of claim 2, wherein the solid support is:
a non-porous substrate or a porous substrate;
a rigid substrate or an elastomeric substrate;
a single-layered substrate or a multilayered substrate, wherein the solid support allows covalent or non-covalent attachment of nucleic acid sequences to its surface; and/or
a substrate with a flat surface without or with chemically or photo etched microwell or nanowell structures, wherein nucleic acid sequences are attached to the flat surface or inner surfaces of microwells or nanowells.

4. The method of claim 2, wherein the nucleic acid features in the template microarray are selected from the group consisting of clonal DNA clusters, DNA nanoballs, DNA coated beads, DNA spots deposited or synthesized by spotting methods, and DNA spots synthesized by light-directed synthesis methods.

5. The method of claim 2, wherein the plurality of nucleic acid features form a random array or a patterned array.

6. The method of claim 2, wherein the synthesis of single-stranded DNA sequences in the template microarray is performed by a method selected from the group consisting of chemical synthesis, enzymatic synthesis, template-dependent synthesis, template-independent synthesis, synthesis of double-stranded DNA sequences followed by cleaving of one or two strands of double-stranded DNAs.

7. The method of claim 6, wherein the cleaving is performed using:
one or more enzymatic reagents optionally selected from the group consisting of uracil-specific excision enzymes, 8-oxoguanine DNA glycosylases, and restriction enzymes;
or one or more chemical reagents, optionally selected from the group consisting of oxidizing reagents and reducing reagents.

8. The method of claim 7, wherein the oxidizing reagent is selected from periodates and lead tetraacetate and the reducing reagent is selected from phosphines, dithiothreitol, dithioerythritol, and L-glutathione.

9. The method of claim 1, wherein the copy microarray comprises an elastomeric solid support having a top surface and a plurality of nucleic acid primer sequences on the top surface.

10. The method of claim 9, wherein the elastomeric solid support is:
a non-porous substrate or a or porous substrate; or
a single-layered substrate or a multilayered substrate; wherein the elastomeric solid support allows covalent attachment of nucleic acid primer sequences to its surface, conformal contact between two microarray supports, and solid-phase clonal DNA amplification on the support.

11. The method of claim 9, wherein a plurality of nucleic acid primers are mixed and evenly cover the top surface of the elastomeric solid support or cover the top surface of the elastomeric solid support in a patterned distribution.

12. The method of claim 1, wherein during the contacting step, a part or substantially all of the single-stranded DNA sequences in each feature in the template microarray hybridize to primer sequences in a copy microarray to serve as templates to synthesize complementary DNA sequences.

13. The method of claim 1, wherein before removing the template microarray, synthesized complementary DNA sequences on the copy microarray are dissociated from single-stranded DNA sequences on the template microarray by thermal and/or chemical denaturation.

14. The method of claim 1, wherein the amplifying step is the solid-phase clonal DNA amplification that is optionally bridge amplification.

15. The method of claim 1, wherein a plurality of contacting steps are performed sequentially by contacting the same template microarray to a plurality of copy microarrays to fabricate a plurality of replicated copy microarrays with nearly identical feature nucleic acid sequences and distributions.

16. The method of claim 1, wherein a replicated copy microarray can be used as a template microarray for an additional microarray replication process.

17. The method of claim 1, wherein a plurality of contacting steps are performed sequentially by replicating a plurality of different template microarrays to the copy microarray to achieve increased feature density and DNA coverage, and decreased feature size on the copy microarray.

18. The method of claim 1, wherein the nucleic acid sequences comprise a single or a plurality of probe sequences for hybridization to complementary sequences in target nucleic acid molecules in a sample.

19. The method of claim 18, wherein target nucleic acid molecules remain in sectioned tissues where their original cellular and tissue locations do not change or wherein target nucleic acid molecules are released from homogenized cells or tissues where cellular membranes are broken.

20. The method of claim 18, wherein target nucleic acid molecules are endogenous nucleic acids in cells or artificially synthesized nucleic acid tags covalently or non-covalently attached to affinity molecules or incorporated into endogenous nucleic acid sequences.

21. The method of claim 20, wherein an affinity molecule is selected from the group consisting of a binding protein, an antibody, an antigen-binding fragment of an antibody, a nanobody, a monobody, and a nucleic acid aptamer.

22. The method of claim 21, wherein the affinity molecule is used to detect one or any combination of proteins, DNAs, RNAs, small-molecule ligands, protein modifications, DNA modifications, RNA modifications, small-molecule ligand modifications, and molecular complexes present in a sample.

23. The method of claim 18, wherein different probe sequences are present in different features such that their sequences are associated with feature positions in a microarray.

24. The method of claim 18, wherein the same probe or a plurality of different probes are present in substantially all features in a microarray.

25. The method of claim 24, wherein in addition to probe sequences, nucleic acid sequences in a feature comprise a barcoding sequence which is associated with a feature position in a microarray.

26. The method of claim 25, wherein the probe and/or the barcoding sequences are known or unknown during microarray construction, and can be later determined by DNA sequencing or hybridization methods.

27. The method of claim 18, wherein an internal probe sequence in DNAs is exposed at the 3′ end by nucleic acid sequence-specific cleavage methods including, but not limited to, restriction enzyme digestion.

28. The method of claim 1, wherein nucleic acid sequences without any probe sequence are added with a single or a plurality of probe sequences by enzymatic methods that are optionally selected from DNA ligation or primer extension after the microarray replication.

29. The method of claim 1, wherein feature probe and/or barcoding sequences in microarrays replicated from the same template microarray are substantially identical and are determined by feature sequences in the template microarray or by only sequencing in one or a few out of many copy microarrays.

30. The method of claim 1, wherein the template and copy microarrays are used to analyze gene expression and genetic variations of single nucleotide polymorphisms in a sample.

31. The method of claim 1, wherein the template and copy microarrays are used to analyze the binding of transcription factors to probe sequences.

32. The method of claim 1, wherein a microarray is used to spatially barcode nucleic acids, proteins, and/or small-molecule ligands in a tissue placed onto the microarray by incorporating barcoding sequences into in situ synthesized complementary DNAs to endogenous nucleic acids or artificially synthesized nucleic acid tags.

33. The method of claim 1, wherein a spatially barcoded microarray is used to analyze molecular interactions between proteins, nucleic acids, and/or small-molecule ligands in a tissue or homogeneous mixture sample.

34. The method of claim 1, wherein the microarray replication is used to amplify natural or synthetic DNAs and/or genes on a chip to minimize bulk polymerase chain reaction (PCR)-induced distortions.

35. The method of claim 1, wherein nucleic acid templates and primers are covalently attached to the template and copy microarrays, restraining DNA diffusion to achieve sub-micrometer resolution feature replication.

36. The method of claim 1, wherein nucleic acid templates covalently attached to a template microarray have a minimal loss in each contact step, so the template microarray can be repeatedly used for many replication cycles.

37. A method of replicating a template microarray comprising:
providing a template microarray comprising a plurality of clonal DNA clusters where each comprises a plurality identical single-stranded DNA sequences that form a random or patterned array distribution;
contacting the template microarray with a copy microarray in the presence of DNA polymerase and nucleotides to synthesize complementary DNA sequences on the copy microarray;
removing the template microarray;
amplifying synthesized complementary DNA sequences on the copy microarray to form a plurality of DNA clusters with the sequences and distribution substantially identical to those in the template microarray; and
repeating the replicating process with the template microarray and a plurality of copy microarrays to produce a plurality of replicated copy microarrays.

38. The method of claim 37, wherein the template microarray comprises a gel or a substrate with chemically or photo etched microwell or nanowell structures and the copy microarray comprises a gel.

39. The method of claim 38, wherein the gel is selected from the group consisting of a polyacrylamide gel, a hydrogel, and a polydimethylsiloxane gel.

40. The method of claim 39, wherein clonal DNA clusters in the template and copy microarrays are amplified by bridge amplification with different cycle numbers to control DNA density or copy number in clusters and cluster sizes.

41. A method of replicating a template microarray comprising:
providing a template microarray comprising a polyacrylamide gel having a plurality of clonal DNA clusters attached thereto;
contacting the template microarray with a copy microarray comprising a polyacrylamide gel having a plurality of primer sequences attached thereto in the presence of DNA polymerase and nucleotides;
removing the template microarray;
amplifying nucleic acid sequences present on the copy microarray to form a plurality of clonal DNA clusters with the sequences and distribution substantially identical to those in the template microarray; and
repeating the replicating process with the template microarray and a plurality of copy microarrays to produce a plurality of replicated copy microarrays.

42. The method of claim 41, wherein the polyacrylamide gel comprises single-layered or multilayered linear polyacrylamide and/or the polyacrylamide crosslinked with one or a plurality of crosslinkers that is optionally selected from the group consisting of N,N′-methylene-bis-acrylamide, N,N′-cystamine-bis-acrylamide, and N,N′-diallyltartardiamide.

43. A method of generating a replicated copy microarray on a gel suitable for electroblotting assays for tissue or homogeneous mixture samples comprising:
replicating a template microarray by the method of claim 1 to a copy microarray on the top surface of a porous gel with the top and bottom surfaces exposed a conducting medium, allowing ions to migrate in or out;
attaching a tissue section or homogeneous mixture sample to the microarray;
applying an electric field vertical to the gel top surface to drive migration of analytes in the sample to the top surface of the gel; and
capturing nucleic acid targets on the gel surface by hybridization to probe sequences in the microarray.

44. The method of claim 43, wherein a sample is a fresh frozen or formalin-fixed paraffin-embedded (FFPE) tissue section or a homogeneous mixture of one or any combination of nucleic acids, proteins, small-molecule ligands, and DNA-tagged affinity molecules.

45. The method of claim 43, wherein the transferring of nucleic acid targets is implemented with a semi-dry or dry electroblotting device.

46. A method of region-selective analysis of nucleic acid targets captured on one or a plurality of regions of a microarray comprising:
replicating a template microarray by the method of claim 1 to a copy microarray having photocleavable probe sequences;
attaching a tissue section or homogeneous mixture sample to the microarray;
capturing nucleic acid targets on the gel surface by hybridization to probe sequences on the microarray;
synthesizing complementary DNA sequences using probe sequences as primers on the microarray;
using light to photocleave probes in one or a plurality of microarray regions to selectively release synthesized complementary DNA sequences; and
eluting released complementary DNAs, wherein the eluted released complementary DNAs are optionally used for DNA amplification, sequencing, and/or other assays.

47. The method of claim 46, wherein probe sequences are linked to a microarray via one or a plurality of photocleavable spacers including, but not limited to, 1-(2-nitrophenyl)ethyl esters.

48. A method of the region-selective analysis of nucleic acid targets captured on one or a plurality of regions of a microarray comprising:
replicating a template microarray by the method of claim 1 to a copy microarray on a gel substrate;
attaching a tissue section or homogeneous mixture sample to the microarray;
capturing nucleic acid targets on the gel surface by hybridization to probe sequences on the microarray;
synthesizing complementary DNA sequences using probe sequences as primers on the microarray;
using a micro cutter device to mechanically cut one or a plurality of gel regions and transfer them to a receptacle that is optionally a tube; and
analyzing complementary DNAs from cut gel pieces by DNA amplification, sequencing, and/or other assays.

49. The method of claim 48, wherein the gel substrate is a non-rigid material suitable for mechanical cutting including, but not limited to, a polyacrylamide gel.

50. The method of claim 48, wherein the micro cutter device has a microdissection head for cutting small gel regions, a tubing for applying vacuum and pressure to the microdissection head to transfer cut gel pieces to a receptacle that is optionally a tube, and an XYZ stage for sampling different gel regions.


SEQUENCE LISTING (28 sequences)

SEQ ID NO: 1 - DNA, 370 bp, synthetic construct
  source 1..370: mol_type = other DNA
aatgatacggcgaccaccgagatctacactgcggccgctaatacgactcactatagggatctnnnnnnnnnnnnnnnnnnnnnnnnagagaatgaggaacccgggaacaatgatggaattttttttttttttttttttcgaaccaccgaggttgccggactagcgcaagtacttgtccattcctgaagaaatattatatttatacaacttacccatagaatcctatttactaggaaaggaaaagcctcctatttataaaaattggatagagctttctcaacaacagtggaatatcaatgatagaacaattgccgatttattagatggggtcttaataataccatcgatctcgtatgccgtcttctgcttg

SEQ ID NO: 2 - protein, 15 aa, synthetic construct
GLNDIFEAQKIEWHE

SEQ ID NO: 3 - protein, 26 aa, synthetic construct
KRRWKKNFIAVSAANRFKKISSSGAL

SEQ ID NO: 4 - protein, 8 aa, synthetic construct
DYKDDDDK

SEQ ID NO: 5 - protein, 9 aa, synthetic construct
YPYDVPDYA

SEQ ID NO: 6 - protein, 6 aa, synthetic construct
HHHHHH

SEQ ID NO: 7 - protein, 10 aa, synthetic construct
EQKLISEEDL

SEQ ID NO: 8 - protein, 15 aa, synthetic construct
KETAAAKFERQHMDS

SEQ ID NO: 9 - protein, 38 aa, synthetic construct
MDEKTTGWRGGHVVEGLAGELEQLRARLEHHPQGQREP

SEQ ID NO: 10 - protein, 13 aa, synthetic construct
SLAELLNAGLGGS

SEQ ID NO: 11 - protein, 8 aa, synthetic construct
TQDPSRVG

SEQ ID NO: 12 - protein, 14 aa, synthetic construct
GKPIPNPLLGLDST

SEQ ID NO: 13 - protein, 8 aa, synthetic construct
DLYDDDDK

SEQ ID NO: 14 - protein, 16 aa, synthetic construct
TDKDMTITFTNKKDAE

SEQ ID NO: 15 - protein, 13 aa, synthetic construct
AHIVMVDAYKPTK

SEQ ID NO: 16 - protein, 8 aa, synthetic construct
WSHPQFEK

SEQ ID NO: 17 - DNA, 39 bp, synthetic construct
  source 1..39: mol_type = other DNA
  modified_base 33: mod_base = OTHER; note = Uracil
ttttttttttaatgatacggcgaccaccgagatctacac

SEQ ID NO: 18 - DNA, 34 bp, synthetic construct
  source 1..34: mol_type = other DNA
ttttttttttcaagcagaagacggcatacgagat

SEQ ID NO: 19 - DNA, 29 bp, synthetic construct
  source 1..29: mol_type = other DNA
  modified_base 23: mod_base = OTHER; note = Uracil
aatgatacggcgaccaccgagatctacac

SEQ ID NO: 20 - DNA, 24 bp, synthetic construct
  source 1..24: mol_type = other DNA
caagcagaagacggcatacgagat

SEQ ID NO: 21 - DNA, 27 bp, synthetic construct
  source 1..27: mol_type = other DNA
cttgcgctagtccggcaacctcggtgg

SEQ ID NO: 22 - DNA, 53 bp, synthetic construct
  source 1..53: mol_type = other DNA
ctacacgacgctcttccgatctnnnnnnnccgctaatacgactcactataggg

SEQ ID NO: 23 - DNA, 22 bp, synthetic construct
  source 1..22: mol_type = other DNA
cctacacgacgctcttccgatc

SEQ ID NO: 24 - DNA, 31 bp, synthetic construct
  source 1..31: mol_type = other DNA
ttccatcattgttcccgggttcctcattctc

SEQ ID NO: 25 - DNA, 23 bp, synthetic construct
  source 1..23: mol_type = other DNA
aagcagtggtatcaacgcagagt

SEQ ID NO: 26 - DNA, 28 bp, synthetic construct
  source 1..28: mol_type = other DNA
  misc_feature 1..25: note = DNA
  misc_feature 26..28: note = RNA
  modified_base 27: mod_base = OTHER; note = Locked nucleic acid
aagctggtatcaacgcagagtgaatggg

SEQ ID NO: 27 - DNA, 58 bp, synthetic construct
  source 1..58: mol_type = other DNA
aatgatacggcgaccaccgagatctacactctttccctacacgacgctcttccgatct

SEQ ID NO: 28 - DNA, 39 bp, synthetic construct
  source 1..39: mol_type = other DNA
caagcagaagacggcatacgagatgtctcgtgggctcgg